SEQUENCE LISTING (20 sequences)

SEQ ID NO: 1 - DNA, 20 bp, synthetic construct
  source 1..20: mol_type = other DNA
acactttctatggctgcccc

SEQ ID NO: 2 - DNA, 20 bp, synthetic construct
  source 1..20: mol_type = other DNA
gtaatgcgtccaggaagcct

SEQ ID NO: 3 - DNA, 20 bp, synthetic construct
  source 1..20: mol_type = other DNA
ggcatcatcaccaactggga

SEQ ID NO: 4 - DNA, 20 bp, synthetic construct
  source 1..20: mol_type = other DNA
ttctcctggcaaagacggac

SEQ ID NO: 5 - DNA, 21 bp, synthetic construct
  source 1..21: mol_type = other DNA
tcgtgcgtgacatcaaagaga

SEQ ID NO: 6 - DNA, 20 bp, synthetic construct
  source 1..20: mol_type = other DNA
tggctgccttcctcttcttg

SEQ ID NO: 7 - DNA, 20 bp, synthetic construct
  source 1..20: mol_type = other DNA
cagctcctacattgggtccc

SEQ ID NO: 8 - DNA, 20 bp, synthetic construct
  source 1..20: mol_type = other DNA
atgccacctcctgagatcct

SEQ ID NO: 9 - DNA, 20 bp, synthetic construct
  source 1..20: mol_type = other DNA
ctgttccagccatccttcat

SEQ ID NO: 10 - DNA, 20 bp, synthetic construct
  source 1..20: mol_type = other DNA
agcactgtgttggcgtacag

SEQ ID NO: 11 - DNA, 20 bp, synthetic construct
  source 1..20: mol_type = other DNA
gtttcagggtccaggtcagg

SEQ ID NO: 12 - DNA, 20 bp, synthetic construct
  source 1..20: mol_type = other DNA
gggggccatcatggtatctg

SEQ ID NO: 13 - DNA, 20 bp, synthetic construct
  source 1..20: mol_type = other DNA
agaggcatagagggacagca

SEQ ID NO: 14 - DNA, 20 bp, synthetic construct
  source 1..20: mol_type = other DNA
ctcaaggtcacggtcacgaa

SEQ ID NO: 15 - DNA, 21 bp, synthetic construct
  source 1..21: mol_type = other DNA
gccacaggattccatacccaa

SEQ ID NO: 16 - DNA, 20 bp, synthetic construct
  source 1..20: mol_type = other DNA
gaaaagcatgagccagcagg

SEQ ID NO: 17 - DNA, 20 bp, synthetic construct
  source 1..20: mol_type = other DNA
ctgggcaggtctactttggg

SEQ ID NO: 18 - DNA, 20 bp, synthetic construct
  source 1..20: mol_type = other DNA
attctccccctgccaaatgg

SEQ ID NO: 19 - DNA, 20 bp, synthetic construct
  source 1..20: mol_type = other DNA
ggcaatgccagggtacatag

SEQ ID NO: 20 - DNA, 20 bp, synthetic construct
  source 1..20: mol_type = other DNA
actcttccagccttccttcc

(19) United States
(12) Patent Application Publication
(10) Pub. No.: US 2024/0425816 A1
(43) Pub. Date: Dec. 26, 2024

(54) COMPOSITIONS AND METHODS OF USE THEREOF FOR TREATING LIVER FIBROSIS

(71) Applicant: Evia Life Sciences Inc., Henderson, NV (US)

(72) Inventors: Takahiro Ochiya, Tokyo (JP); Kiminori Kimura, Tokyo (JP); Juntaro Matsuzaki, Tokyo (JP)

(21) Appl. No.: 18/702,748

(22) PCT Filed: Oct. 18, 2022
(86) PCT No.: PCT/US2022/078303

§ 371 Date: Apr. 18, 2024

Related U.S. Application Data
(60) Provisional application No. 63/256,840, filed on Oct. 18, 2021.

(51) Int. Cl. C12N 5/071; A61K 9/50; A61K 38/19; A61P 1/16; C12N 15/113

(52) U.S. Cl. CPC C12N 5/0672; A61K 9/5068; A61K 38/191; A61P 1/16; C12N 15/113; C12N 2310/141; C12N 2501/727; C12N 2506/14

(57) ABSTRACT

Compositions, methods of making compositions, and methods of using compositions for treatment of liver diseases, disorders, and damage are provided. The compositions can include chemically induced liver progenitor cells (CLiPs), and/or cell-free materials formed from the CLiPs such as extracellular vesicles (EVs) such as exosomes. In some embodiments, the compositions and/or methods are effective to reduce existing hepatic collagen or the formation of new hepatic collagen; and/or reduce the amount of existing fibrosis or the formation of new fibrosis in a subject in need thereof.

### CROSS-REFERENCE TO RELATED APPLICATION

This application claims the benefit of and priority to U.S. Ser. No. 63/256,840 filed Oct. 18, 2021, which is specifically incorporated by reference herein in its entirety.

### REFERENCE TO SEQUENCE LISTING

The Sequence Listing submitted as an xml file named “EVIA102PCT.xml,” created on Oct. 18, 2022, and having a size of 18,496 bytes is hereby incorporated by reference pursuant to 37 C.F.R. § 1.834(c)(1).

### FIELD OF THE INVENTION

The field of the invention generally relates to chemically induced liver progenitor cells, and compositions made therewith and therefrom, and methods of using the same for treating liver fibrosis.

### BACKGROUND OF THE INVENTION

Hepatocytes are regarded as the only effective cell source for cell transplantation to treat liver diseases; however, their availability is limited due to a donor shortage. Thus, improved cell sources and alternative treatments must be developed. Results show that hepatic progenitor cells with repopulative capacity can be obtained from mature rodent hepatocytes and human infant hepatocytes using the proper combination of small molecule inhibitors (Katsuda et al., Cell Stem Cell 20, 41-55, (2017), dx.doi.org/10.1016/j.stem.2016.10.007, Katsuda et al., eLife 8:e47313, 31 pages, (2019) doi.org/10.7554/eLife.47313). However, the mechanisms underlying the ability of these cells to improve liver function and thus range of their use for treating liver diseases and disorders remained unclear.

Thus, it is object of the invention to provide insight into the mechanisms underlying the ability of chemically induced liver progenitor cells to improve liver function, and improved compositions and methods of use thereof developed based thereon.

### SUMMARY OF THE INVENTION

Compositions, methods of making compositions, and methods of using compositions for treatment of liver diseases, disorders, and damage are provided. The compositions include chemically induced liver progenitor cells (CLiPs), and/or cell-free materials formed from the CLiPs such as extracellular vesicles (EVs) such as exosomes. In some embodiments, the compositions and/or methods are effective to reduce existing hepatic collagen or the formation of new hepatic collagen; reduce the amount of existing fibrosis or the formation of new fibrosis; induce a change in expression of one or more hepatic fibrosis-associated genes, optionally increase expression of Mmp2 mRNA, reduce expression of Timp1, αSMA, and/or Col1a mRNA and/or protein, or any combination thereof; induce a reduction in the expression of one or more markers of hepatic stellate cell activation, such as αSMA, preferrable in hepatic stellate cells; induce a change in the expression of one or more genes associated with cell cycle, autophagy, cell membrane fusion, and/or zinc finger protein, optionally wherein the gene(s) is Dmtf1, Zfp612, Itga6, Trim24, Eaf2, Zfp119a, Dido1, Masp2, Sgk1, Sm11567, Eml5, Srsf5, Rab35, Fam206a, Zfp131, Zkscan14, Insc, Ntn3, or a combination thereof; induce an increase in MMP1 and/or MMP13 mRNA and/or protein in hepatic stellate cells; and/or induce a reduction in TNFα mRNA and/or protein in hepatic stellate cells in a subject in need thereof.

Methods of making EVs formed from CLiPs are also provided. The methods typically include culturing CLiPs and harvesting EVs secreted by the CLiPs. Typically, the cells are cultured with an inhibitor of TGFβ signaling, such as A83-01, for example at a concentration of about 1 μM to about 10 μM, or about 0.1 μM to about 10 μM, or about 0.5 μM. Typically, the cells are also cultured with a GSK3 inhibitor, such as CHIR99021, for example at a concentration of about 0.1 μM to about 20 μM, about 1 μM to about 10 μM, or about 3 μM.

Typically, the cells begin as hepatocytes isolated/purified from a liver of a mammal.

In some embodiments, particularly those in which the starting cells are human hepatocytes, the cells are cultured with serum, such as Fetal Bovine Serum (FBS), for example at a concentration of 5-20% of the culture media, or about 10% of the culture media.

In some embodiments, particularly those in which the starting cells are rodent cells such as from mouse or rat, the cells are cultured with a ROCK inhibitor, such as Y-27632, for example at a concentration of about 1 μM to about 100 μM, or about 5 μM to about 25 μM, or about 10 μM. In some embodiments, particularly those in which the cells are human, ROCK inhibitor may be excluded from the culturing.

The cells can be cultured with the inhibitor(s) and/or serum for at least 5, 6, 7, 8, 9, 10, 11, 12, 13, 14, 15, 16, 17, 18, 19, or 20 days; or about 5 days to about 25 days, or any subrange or integer number of days therebetween, optionally for about 7 days to about 22 days, about 5 days to about 25 days, or about 10 days to about 20 days, or about 12 days to about 17 days; or about 13, 14, or 15 days.

Pharmaceutical compositions including an effective amount of CLiPs and/or EVs formed therefrom are also provided.

The compositions can be used in therapeutic and non-therapeutic methods of treating a subject in need thereof that typically includes administering the subject a pharmaceutical composition including an effective amount of CLiPs and/or EVs formed therefrom. In some embodiments, the method is effective for treating a subject for liver fibrosis.

In some embodiments, the composition includes EVs or CLiPs that secrete EVs, the EVs having one or more microRNAs, for example one or more of hsa-miR-103a-3p, hsa-miR-hsa-miR-122-5p, hsa-miR-125a-5p, hsa-miR-125b-5p, hsa-miR-126-3p, hsa-miR-1324, hsa-miR-142-3p, hsa-miR-151a-3p, hsa-miR-155-5p, hsa-miR-16-5p, hsa-miR-182-5p, hsa-miR-183-5p, hsa-miR-191-5p, hsa-miR-192-5p, hsa-miR-21-5p, hsa-miR-221-3p, hsa-miR-224-5p, hsa-miR-23a-3p, hsa-miR-24-3p, hsa-miR-24-3p, hsa-miR-26a-3p, hsa-miR-28-3p, hsa-miR-29a-3p, hsa-miR-29b-3p, hsa-miR-30a-5p, hsa-miR-30d-5p, hsa-miR-30e-5p, hsa-miR-31-5p, hsa-miR-34a-5p, hsa-miR-3663-3p, hsa-miR-4435, hsa-miR-4440, hsa-miR-5096, hsa-miR-510-3p, hsa-miR-92a-3p, hsa-miR-93-5p, and hsa-miR-99b-5p, and/or one or more cytokines optionally wherein the cytokine(s) is or includes TNFα, or any combination thereof.

In some embodiments, the methods further include administering the subject a second active agent. In some embodiments, the methods include administering the subject TNFα.

The methods can be used to treat subjects with a liver disease or disorder, such as an infection, optionally hepatitis A, B, or C; an immune system problem, optionally autoimmune hepatitis, primary biliary cholangitis, or primary sclerosing cholangitis; a cancer optionally liver cancer, bile duct cancer, or liver cell adenoma; an inherited liver disorder, optionally hemochromatosis, hyperoxaluria, Wilson's disease, or Alpha-1 antitrypsin deficiency; damage from alcohol abuse and/or drug overdose; or nonalcoholic fatty liver disease.

### BRIEF DESCRIPTION OF THE DRAWINGS

FIGS. 1A and 1B are line graphs showing blood biochemical test results in mouse model for hepatic fibrosis. As of the transplantation, the total bilirubin value showed a normal value, and the platelet count was higher than the reference value (Table 2). AST (FIG. 1A) and ALT (FIG. 1B) were confirmed over time after the transplantation, finding no significant difference between the transplantation group and the non-transplantation group.

FIG. 2 is a plot showing collagen amount in the liver tissue with and without hCLiP transplantation.

FIG. 3A is a plot showing Col1a positive area in the liver tissue with and without hCLiP transplantation. FIG. 3B is plot showing the change in pathological hepatic fibrosis through hCLiP transplantation.

FIGS. 4A-4D are bar graphs showing the change in the expression of the hepatic fibrosis-associated genes: Mmp1 mRNA (FIG. 4A), Timp1 mRNA (FIG. 4B), Col1a mRNA (FIG. 4C), αSMA mRNA (FIG. 4D) due to hCLiP transplantation.

FIG. 5 is a plot showing the presence of hCLiPs in the liver tissue. Total DNA was collected from frozen liver tissue, and Mouse Tfrc and Human RNase P were used to measure the copy number of each of them. 0-1% of human cells were detected in the transplantation group.

FIG. 6 is a heat map showing the change in the gene profile due to hCLiP transplantation. hCLiP transplantation resulted in a significant decrease in expression of 18types of genes.

FIG. 7 is a bar graph showing the change in the hepatic stellate cell activation level by co-culture of hepatic stellate cells and hCLiPs.

FIGS. 8A-8D are bar graphs showing CYP3A4 enzyme activity by induction of hepatic differentiation of immortalized hCLiPs “A”-“D” in FIGS. 8A-8D, respectively.

FIG. 9 is a bar graph showing the change in the hepatic stellate cell activation level by co-culture of hepatic stellate cells and immortalized hCLiPs

FIGS. 10A-10D are bar graphs showing the change in the gene expression: TNFα mRNA (FIG. 10A), TIMP3 mRNA (FIG. 10B), MMP13 mRNA (FIG. 10C), and MMP1 mRNA (FIG. 10D) in hepatic stellate cells due to co-culture of hepatic stellate cells and hCLiPs.

FIGS. 11A-11C are bar graphs showing the change of hCLiPs gene expression: MMP13 mRNA (FIG. 11A), TIMP3 mRNA (FIG. 11B), and TNFα mRNA (FIG. 11C), due to co-culture of hepatic stellate cells and hCLiPs in the presence of TGF.

FIG. 12 is a bar graph showing the change in the gene expression: αSMA mRNA upon addition of 10, 20, or 50 ng/ml of TNFα to hepatic stellate cells.

FIGS. 13A and 13B are bar graphs showing the change in the hepatic stellate cell activation level due to addition of a hCLiP-derived exosome to hepatic stellate cells. FIG. 13A shows the change in αSMA, which is a hepatic stellate cell activation marker, at the protein level in hepatic stellate cells with and without exosomes, and with and without TGFβ. FIG. 13B shows levels of mRNA (MMP13, TIMP3, IL-13, and TNFα) in the added exosomes.

FIGS. 14A-14C are bar graphs showing mRNA: MMP13 (FIG. 14A), TIMP3 (FIG. 14B), and TNFα (FIG. 14C) in a hCLiP-derived exosome in the presence or absence of TGFβ. n=1.

FIG. 15 is a model illustrating a proposed action mechanism explaining the hCLiPs-induced improvement in hepatic fibrosis. Proposed roles for TNFα, which is a hCLiP-derived cytokine, and hCLiP-derived exosomes are shown.

FIG. 16 is a bar graph showing the relative levels of various microRNAs detected in hCLiP EVs. Circled miRNAs indicate those that may be particularly influential in inhibiting liver fibrosis.

### DETAILED DESCRIPTION OF THE INVENTION

### I. DEFINITIONS

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, “substantially changed” means a change of at least e.g. 5%, 10%, 15%, 20%, 25%, 30%, 35%, 40%, 45%, 50%, 75%, 100%, or more relative to a control.

As used herein, the term “purified,” “isolated,” and like terms relate to the isolation of a molecule or compound in a form that is substantially free (at least 60% free, preferably 75% free, and most preferably 90% free) from other components normally associated with the molecule or compound in a native environment.

As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### II. COMPOSITIONS

Disclosed herein are compositions and methods for treating liver diseases, disorders, and injuries. The compositions can include and/or or be formed by chemically induced liver progenitor cells (CLiPs). The compositions can be cell-based compositions, or cell-free compositions. Methods of making CLiPs are also provided.

### A. Chemically Induced Liver Progenitor Cells (CLiPs)

The disclosed compositions and methods are typically composed of or formed from chemically induced liver progenitor cells (CLiPs), preferably human chemically induced liver progenitor cells (hCLiPs). The cells are preferably not intentionally genetically modified, for example, modified by recombinant genetic technology, directed gene editing, etc. However, genetically modified cells are also contemplated. Examples include, but are not limited, to immortalized CLiPs such as those having CDK4, CCND1 (cyclin D1), and/or TERT, typically under the control of a conditional or constitutively active promoter (see, e.g., the Examples below).

### 1. Sources of Starting Hepatocytes

The liver cells, also referred to as hepatocytes, used as a starting material for chemical induction typically include at least one type of hepatocyte marker genes (for example, albumin (ALB), transthyretin (TTR), glucose-6-phosphatase (G6PC), tyrosine aminotransferase (TAT), tryptophane-2,3-dioxygenase (TDO2), cytochrome P450 (CYP), miR-122, etc.), preferably 2 or more types, more preferably 3 or more types, still more preferably 4 or more types, particularly preferably 5 or more types, and most preferably all of the 6 types selected from ALB, TTR, G6PC, TAT, TDO2 and CYP. Preferably, the hepatocytes are functional. Functional hepatocytes refers to hepatocytes that retain one or more, preferably 2 or more, more preferably 3 or more, still more preferably 4 or more, and most preferably all of the functions selected from: (i) having a bile canalicular structure and accumulating drug metabolites in the canaliculi; (ii) expressing ABC transporters (e.g., MDR1, MRP, etc.) in the cell membrane; (iii) secretorily expressing ALB; (iv) accumulating glycogen; and (v) having activity as a drug-metabolizing enzyme (e.g., CYP1A1, CYP1A2, etc.).

The hepatocytes can be provided from any source as long as they are hepatocytes, e.g., as characterized by the expression of the above-described hepatocyte marker genes. For example, the hepatocytes can be obtained from a mammal, for example, a human, rat, mouse, guinea pig, rabbit, sheep, horse, pig, bovine, monkey or the like, preferably human, rat or mouse. The hepatocytes can be obtained from embryonic stem cells (ES cells) or pluripotent stem cells such as iPS cells by a differentiation inducing method, or from fibroblasts by direct reprogramming. In some embodiments, the hepatocytes are free from genetic modification.

An exemplary source is hepatocytes isolated/purified from a liver of a mammal. For example, in the case of a non-human mammal, a liver can be removed. For human, an adult liver tissue piece can be sectioned by surgical operation, or from a recently deceased donor which may be an adult or juvenile. A liver sectioned from the fetus of a terminated pregnancy may also be used. Cells can be freshly isolated or can be cryopreserved cells that are isolated/purified hepatocytes previously removed from a liver. The liver can be a heathy liver. In some embodiments, the liver cells are autologous to the subject to be treated.

Hepatocytes can be purified from a mammal liver or a tissue piece thereof by a perfusion method (“Handbook of Cultured Cell Experiments” (Yodosha, 2004), etc.). Specifically, following pre-perfusion with an EGTA solution via the portal vein, a liver can be digested by perfusion with an enzyme solution (Hank's solution, etc.) such as collagenase or dispase, the hepatocytes can be purified by removing cell pieces and non-parenchymal cells by filtration, low-speed centrifugation or the like.

### 2. Inhibitors, Serum, and Other Factors

To form CLiPs, the hepatocytes are typically contacted with one or more inhibitors of TGF-β receptor and one or more inhibitors of GSK3. In some embodiments, the cells are also brought into contact with one or more ROCK inhibitors and/or a serum. The contacting typically occurs in vitro/ex vivo.

Each of the inhibitors, discussed in more detail below, can be a protein, nucleic acid, small molecule, antibody or other agent that reduces or prevents expression of the target molecule or signaling pathways (e.g., TGF-beta, GSK3, ROCK, etc.).

Inhibitors can directly or indirectly inhibit or otherwise reduce expression or activity of the target molecule. For example, negative regulators of ROCK activation include small GTP-binding proteins such as Gem, RhoE, and Rad, which can attenuate ROCK activity. Auto-inhibitory activity of ROCK has also been demonstrated upon interaction of the carboxyl terminus with the kinase domain to reduce kinase activity.

Inhibitors can be, but are not limited to, small molecules, antibodies, antisense compounds and negative regulators. Preferably one or more or all of the inhibitors are low molecular weight compounds (e.g., small molecules).

In other examples, the inhibitor is an antisense compound. Generally, the principle behind antisense technology is that an antisense compound hybridizes to a target nucleic acid and effects the modulation of gene expression activity, or function, such as transcription, translation or splicing. The modulation of gene expression can be achieved by, for example, target RNA degradation or occupancy-based inhibition. An example of modulation of target RNA function by degradation is RNase H-based degradation of the target RNA upon hybridization with a DNA-like antisense compound, such as an antisense oligonucleotide. Antisense oligonucleotides can also be used to modulate gene expression, such as splicing, by occupancy-based inhibition, such as by blocking access to splice sites.

Antisense compounds include, but are not limited to, antisense oligonucleotides, siRNA, miRNA, shRNA and ribozymes. Antisense compounds can specifically target a nucleic acid encoding the target for inhibition. Each of the above-described antisense compounds provides sequence-specific target gene regulation. This sequence-specificity makes antisense compounds effective tools for the selective modulation of a target nucleic acid of interest. Methods of designing, preparing and using antisense compounds that specifically target nucleic acids are within the abilities of one of skill in the art.

In another embodiment, the inhibitor can be a function blocking antibody.

a. TGF-β Receptor Inhibitor

The hepatocytes are typically brought into contact with one or more low-molecular weight signaling pathway inhibitors including a TGF-β receptor inhibitor in vitro. The TGF-β receptor inhibitor used for the present invention may be any inhibitor as long as it inhibits the function of the transforming growth factor (TGF)-β receptor, and including inhibitors of TGF-beta/Smad signaling such as small molecules, antibodies, antisense compounds and negative regulators of TGF-beta/Smad signaling molecules. Antibodies, antisense compounds and negative regulators can be designed to target TGF-β signaling molecules such as ALK4, 5, and/or 7.

Exemplary small molecule inhibitors of TGF-β/Smad signaling include, but are not limited to, A83-01, SB431542, LDN-193189, Galunisertib (LY2157299), LY2109761, SB525334, SB505124, GW788388, LY364947, RepSox (E-616452), LDN-193189 2HCl, K02288, BIBF-0775, TP0427736 HCl, LDN-214117, SD-208, Vactosertib (TEW-7197), ML347, LDN-212854, DMH1, Dorsomorphin (Compound C), 2HCl, Pirfenidone (S-7701), Sulfasalazine (NSC 667219), AUDA, PD 169316, TA-02, ITD-1, LY 3200882, Alantolactone, Halofuginone, SIS3 HCl, Dorsomorphin (Compound C), and Hesperetin.

Other examples include, but are not limited to, 2-(5-benzo[1,3]dioxole-4-yl-2-tert-butyl-1H-imidazol-4-yl)-6-methylpyridine, 3-(6-methylpyridine-2-yl)-4-(4-quinolyl)-1-phenylthiocarbamoyl-1H-pyrazole (A-83-01), 2-(5-chloro-2-fluorophenyl)pteridine-4-yl)pyridine-4-ylamine (SD-208), 3-(pyridine-2-yl)-4-(4-quinonyl)]-1H-pyrazole, 2-(3-(6-methylpyridine-2-yl)-1H-pyrazole-4-yl)-1,5-naphthyridine (all from Merck) and SB431542 (Sigma Aldrich).

A preferred example is A-83-01, also referred to herein as “A”. Typically, for example, the inhibitor A83-01 is used in a concentration of about 0.1 μM to about 10 μM, or about 0.5 μM.

b. GSK3 Inhibitor

The hepatocytes are also typically brought into contact with one or more inhibitors of GSK3 inhibitor in vitro. The GSK3 inhibitor can be any GSK3 inhibitor as long as it inhibits the function of glycogen synthase kinase (GSK) 3. Examples include SB216763 (Selleck), CHIR98014, CHIR99021 (all from Axon medchem), SB415286 (Tocris Bioscience), and Kenpaullone (Cosmo Bio). A preferred example is CHIR99021, also referred to herein as “C”. Typically, for example, the inhibitor CHIR99021 is used in a concentration of about 0.1 μM to about 20 μM, about 1 μM to about 10 μM, or about 3 μM.

c. ROCK Inhibitor

In some embodiments, the hepatocytes are also brought into contact with one or more inhibitors of ROCK. In some embodiments, for example when the hepatocytes are human cells, the ROCK inhibitor may be excluded.

Rho-associated kinase (also known as and/or referred to herein as ROCK, Rock, Rho-associated coiled-coil kinase, and Rho kinase, includes ROCK1 (also called ROKβ or p160ROCK) and ROCK2 (also called ROKα). ROCK proteins are serine-threonine kinases that interact with Rho GTPases. Preferably, the ROCK inhibitor is a small molecule. Exemplary small molecule ROCK inhibitors include Y-27632 (U.S. Pat. No. 4,997,834) and fasudil (also known as HA 1077; Asano et al., J. Pharmacol. Exp. Ther. 241:1033-1040, 1987). These inhibitors bind to the kinase domain to inhibit ROCK enzymatic activity. Other small molecules reported to specifically inhibit ROCK include H-1152 ((S)-(+)-2-Methyl-1-[(4-methyl-5-isoquinolinyl)sulfonyl]homopiperazine, Ikenoya et al., J. Neurochem. 81:9, 2002; Sasaki et al., Pharmacol. Ther. 93:225, 2002); N-(4-Pyridyl)-N′-(2,4,6-trichlorophenyl)urea (Takami et al., Bioorg. Med. Chem. 12:2115, 2004); and 3-(4-Pyridyl)-1H-indole (Yarrow et al., Chem. Biol. 12:385, 2005), GSK269962A (Axon medchem), and Fasudil hydrochloride (Tocris Bioscience).

Additional small molecule Rho kinase inhibitors include those described in PCT Publication Nos. WO 03/059913, WO 03/064397, WO 05/003101, WO 04/112719, WO 03/062225 and WO 03/062227; U.S. Pat. Nos. 7,217,722 and 7,199,147; and U.S. Patent Application Publication Nos. 2003/0220357, 2006/0241127, 2005/0182040 and 2005/0197328.

In particularly preferred embodiments, the ROCK inhibitor is Y-27632, also referred to herein as “Y”. Also known as (+/−)-trans-N-(4-Pyridyl)-4-(1-aminoethyl)-cyclohexanecarboxamide, Y-27632 is a small molecule inhibitor that selectively inhibits activity of Rho-associated kinase. Y-27632 is disclosed in U.S. Pat. No. 4,997,834 and PCT Publication No. WO 98/06433. In some embodiments, when the ROCK inhibitor is Y-27632, the effective amount of the ROCK inhibitor is about 1 to about 100 μM, or about 5 to about 25 μM, or about 10 μM.

The GSK3 inhibitor and the ROCK inhibitor hardly induce hepatic stem/progenitor cells when they are individually brought into contact with hepatocytes, whereas efficiency of inducing hepatic stem/progenitor cells (also referred to as “reprogramming efficiency”) is significantly increased when the GSK3 inhibitor together with the TGF-β receptor inhibitor are brought into contact with the hepatocytes as compared to the case where only the TGF-β receptor inhibitor is brought into contact with the hepatocytes. In addition, reprogramming efficiency of rat and mouse cells is also increased when the ROCK inhibitor together with the TGF-β receptor inhibitor are brought into contact with the hepatocytes as compared to a case where only the TGF-β receptor inhibitor is brought into contact with the hepatocytes (Katsuda et al., Cell Stem Cell 20, 41-55, (2017), dx.doi.org/10.1016/j.stem.2016.10.007, which is specifically incorporated by reference herein in its entirety. Therefore, in some embodiments, a GSK3 inhibitor and/or the ROCK inhibitor, in addition to the TGF-β receptor inhibitor, is brought into contact with the hepatocytes.

d. Serum and Other Factors

Results also show that when using some human hepatocytes, such as infant primary human hepatocytes (IPHHs), the cells are preferably not contacted with a ROCK inhibitor, and additionally or alternatively are contacted with a serum, such as fetal bovine serum (Katsuda et al., eLife 8:e47313, 31 pages, (2019) doi.org/10.7554/eLife.47313, which is specifically incorporate by reference herein in its entirety). Thus, in some embodiments, a GSK3 inhibitor and/or a serum, in addition to the TGF-β receptor inhibitor, is brought into contact with the hepatocytes.

Examples of serum include those from mammals including, but not limited to, bovines, humans, horses, goats, rabbits, sheep, pigs, rats, and mice. In particular embodiments, the serum is Fetal Bovine Serum (FBS), Fetal or neonatal Calf Serum (FCS), Adult Bovine Serum (ABS), and Human Serum. When present, serum is typically present as 5-20% of the culture media. In a particular embodiment, the serum is 10% FBS.

In a case of a serum-free medium, a serum substitute (BSA, HAS, KSR, etc.) may be added.

In general, factors such as a growth factor, cytokine, or hormone are further added. Examples of such factors include, but not limited to, one or more of epidermal growth factor (EGF), insulin, transferrin, hepatocyte growth factor (HGF), oncostatin M (OsM), hydrocortisone 21-hemisuccinate or a salt thereof and dexamethasone (Dex).

e. MEK Inhibitors

A low-molecular weight signaling pathway inhibitor other than the GSK3 inhibitor and the ROCK inhibitor may also be combined with the TGF-β receptor inhibitor. An example of such an inhibitor includes, but not limited to, a MEK inhibitor. The MEK inhibitor is not particularly limited and any inhibitor may be used as long as it inhibits the function of MEK (MAP kinase-ERK kinase), where examples include AZD6244, CI-1040 (PD184352), PD0325901, RDEA119 (BAY869766), SL327, U0126 (all from Selleck), PD98059, U0124 and U0125 (all from Cosmo Bio).

f. Exemplary Preferred Embodiments

In particular, it is preferable to contact the cells with at least:

A-83-01 (A) as the TGF-β receptor inhibitor in combination with CHIR99021 (C) as the GSK3 inhibitor (AC), optionally in further combination with a serum, e.g., FBS (FAC);

A-83-01 (A) as the TGF-β receptor inhibitor in combination with Y-27632 (Y) as the ROCK inhibitor (YA), optionally in further combination with a serum, e.g., FBS (FYA);

A-83-01 (A) as the TGF-β receptor inhibitor in combination with CHIR99021 (C) as the GSK3 inhibitor and Y-27632 (Y) as the ROCK inhibitor (YAC), optionally in further combination with a serum, e.g., FBS (FYAC).

A preferred formulation for culturing mouse and rat hepatocytes is YAC.

A preferred formulation for culture IPHHs is FAC.

In particular embodiments, the concentration of the TGF-β receptor inhibitor added to the medium may suitably be selected, for example, in a range of 0.01-10 μM, and preferably 0.1-1 μM; the concentration of the GSK3 inhibitor added to the medium may suitably be selected, for example, in a range of 0.01-100 μM, and preferably 1-10 μM; the concentration of the ROCK inhibitor added to the medium may suitably be selected, for example, in a range of 0.0001-500 μM, and preferably 1-50 μM; and the concentration of the serum added to the medium may suitably be selected, for example, in a range of 5%-20%, preferably 8%-12%, for example 10%.

Inhibitors and/or methods of making CLiPs are also described in one or more of WO 2020/080550, WO 2017/119512, U.S. Pat. No. 10,961,507, U.S. Ser. No. 17/285,038, Katsuda et al., Cell Stem Cell 20, 41-55, (2017), dx.doi.org/10.1016/j.stem.2016.10.007, and Katsuda et al., eLife 8:e47313, 31 pages, (2019) doi.org/10.7554/eLife.47313 each of which is specifically incorporated by reference herein in its entirety.

### 3. Culturing and Selection Guidelines

Contact between hepatocytes and the inhibitor(s) and optional serum can be carried out by culturing the hepatocytes in the presence of these materials. Specifically, these inhibitor(s) and optionally serum are added to a medium at an effective concentration to carry out the culturing. Examples of suitable media include, but are not limited to, basal medium. A commercially available basal medium may also be employed, where examples include, but are not particularly limited to, a minimum essential medium (MEM), a Dulbecco's modified minimum essential medium (DMEM), a RPMI1640 medium, a 199 medium, a Ham's F12 medium and a William's E medium, which may be used alone or two or more types of them may be used in combination. Examples of additives to the medium include various amino acids (for example, L-glutamine, L-proline, etc.), various inorganic salts (salt of selenious acid, NaHCO₃, etc.), various vitamins (nicotinamide, ascorbic acid derivative, etc.), various antibiotics (for example, penicillin, streptomycin, etc.), an antimycotic agent (for example, amphotericin, etc.), and buffers (HEPES, etc.).

When these inhibitors are water-insoluble or poorly water-soluble compounds, they may be dissolved in a small amount of a low-toxicity organic solvent (for example, DMSO, etc.), and then the resultant can be added to a medium to give the above-described final concentration.

The culture vessel used for this culture is not particularly limited as long as it is suitable for adhesion culture, where examples include a dish, a petri dish, a tissue culture dish, a multidish, a microplate, a microwell plate, a multiplate, a multiwell plate, a chamber slide, a Schale, a tube, a tray, and a culture bag. The culture vessel used may have its inner surface coated with a cell supporting substrate for the purpose of enhancing adhesiveness with the cells. Examples of such a cell supporting substrate include collagen, gelatin, Matrigel, poly-L-lysine, laminin and fibronectin, and is preferably collagen and/or Matrigel.

The hepatocytes can be seeded onto a culture vessel at a cell density of 10²-10⁶ cells/cm², and preferably 10³-10⁵ cells/cm². Culture can take place in a CO₂ incubator, in an atmosphere at a CO₂ concentration of 1-10%, preferably 2-5% and more preferably about 5%, at 30-40° C., preferably 35-37.5° C. and more preferably about 37° C. The culture period may be, for example, 1-4 weeks, preferably 1-3 weeks, and more preferably about 2 weeks. The medium can be freshly exchanged every 1-3 days.

In this manner, the hepatocytes are brought into contact with the TGF-β receptor inhibitor, and optionally the GSK3 inhibitor and/or the ROCK inhibitor and/or serum so as to reprogram the hepatocytes into hepatic stem/progenitor cells. Although mature hepatocytes are generally not considered to proliferate in vitro, they were found to proliferate by about 15 times by 2 weeks of culture with YAC as described in Katsuda et al., Cell Stem Cell 20, 41-55, (2017), dx.doi.org/10.1016/j.stem.2016.10.007. Similarly, IPHHs proliferated efficiently and became the predominant population over 2 weeks in culture with FAC (Katsuda et al., eLife 8: e47313, 31 pages, (2019) doi.org/10.7554/eLife.47313).

In preferred embodiments, the CLiPs have

(a) self-regeneration ability; and

(b) bipotential ability to differentiate into both hepatocytes and biliary epithelial cells. Herein, the term “biliary epithelial cells” (also referred to as “BEC”) refers to cells that express cytokeratin 19 (CK19) and GRHL2 as BEC markers.

The CLiPs may also include fetal liver hepatoblast and oval cells that emerge upon liver damage.

In a preferred embodiment, the features (a) and (b) above and similar to conventionally known liver stem cells (LSC), CLiPs obtained by the disclosed reprogramming method:

(c) express epithelial cell adhesion molecule (EpCAM) as a surface antigen marker but do not express delta homolog 1 (Dlk1) expressed by other known LSC. In addition, according to some embodiments, CLiPs do not express leucine-rich repeat-containing G protein-coupled receptor 5 (LGRS) and FoxL1 which are known LSC markers.

CLiPs can also have one or more of the following features:

(d) the apparent growth rate does not slow down for at least 10 passages, preferably 20 passages or more;

(e) differentiation potency into hepatocytes and BEC is retained for at least 10 passages, preferably 20 passages or more;

(f) nuclear cytoplasmic (N/C) ratio is higher than that of hepatocytes;

(g) expressions of one or more LSC marker genes selected from the group consisting of α-fetoprotein (AFP), SRY-box (Sox) 9, EpCAM, Thy-1/CD90, hepatocyte nuclear factor 1 homeobox B (HNF1-β), forkhead box J1 (FoxJ1), HNF6/one cut-1 (OC1), CD44, integrin α6 (A6) and CK19 gene are increased compared to hepatocytes.

(h) expressions of one or more proteins selected from the group consisting of AFP, CD44, EpCAM, CK19, Sox9, A6 and CD90 are increased compared to hepatocytes.

In some embodiments, CLiPs have all of the above-described features (d)-(h).

Accordingly, CLiPs can be induced from hepatocytes by bringing the hepatocytes into contact, most typically in vitro or ex vivo, with a TGF-β receptor inhibitor, and preferably further with a GSK3 inhibitor and/or a ROCK inhibitor and/or serum in effective amounts and under suitable conditions to induce cells having one or more, preferably most or all, of the features discussed above.

### 4. Maintenance/Proliferation of CLiPs

The CLiPs can be efficiently maintained/proliferated by passaging them in the presence of the inhibitor(s) and optionally serum, e.g.,

(i) on a collagen-or Matrigel-coated culture vessel for the first to fourth passages; and

(ii) on a Matrigel-coated culture vessel for the fifth passage and so forth.

As the culture vessel, a culture vessel similar to one used for inducing CLiPs from hepatocytes can be used. The culture vessels used for the first to fourth passages are coated with collagen or Matrigel.

Once the primary CLiPs obtained as described above reach 70-100% confluency, they can be seeded onto this collagen-or Matrigel-coated culture vessel at a density of 10³-10⁵ cells/cm². As the medium, the medium described for induction culture of CLiPs can similarly be used. The concentrations of the inhibitor(s) and optionally serum added can also suitably be selected from the concentration ranges described above for induction culture of CLiPs. The culture temperature and the CO₂ concentration also follow the conditions for induction culture of CLiPs. Once 70-100% confluency is reached, the cells can be treated with trypsin to be dissociated, and passaged.

For the fifth passage and so forth, a Matrigel-coated culture vessel is preferably used. Stable CLiPs can be obtained after about 5-8 passages. After 10 passages or more, cloning can be conducted by a routine procedure.

As described above, the inhibitor(s) and optionally serum can be added to the medium not only for CLiPs induction culture but also for maintenance/proliferation culture.

5. Redifferentiation from CLiPs into Hepatocytes

In some embodiments, the CLiPs are utilized as CLiPs. In other embodiments, the CLiPs are redifferentiated into hepatocytes. Induction of CLiPs to redifferentiate into hepatocytes may be carried out by any known method. Such method can be, for example, a method of culturing in a culture solution added with oncostatin M (OsM), dexamethasone (Dex), hepatocyte growth factor (HGF) or the like (Journal of Cellular Physiology, Vol. 227(5), p. 2051-2058 (2012); Hepatology, Vol. 45(5), p. 1229-1239 (2007)), or a method combined with a Matrigel overlaying method (Hepatology 35, 1351-1359 (2002)). The medium for inducing differentiation into hepatocytes may or may not be added, but preferably added, with inhibitor(s) and optionally serum.

The hepatocytes obtained by inducing differentiation of CLiPs can have a bile canaliculus-like structure typical of mature hepatocytes, and thus can accumulate drug metabolites in the canaliculi. In addition, they may express an ABC transporter such as MRP2 protein in the cell membrane. Moreover, they may exert a series of hepatic functions such as secretory expression of albumin, glycogen accumulation, and cytochrome p450 (CYP) drug-metabolizing enzyme activity. Specifically, CLiPs can be redifferentiated into functional hepatocytes.

6. Induction of Differentiation of CLiPs into BEC

Induction of differentiation of CLiPs into BEC can be carried out by any known method. Such method can be, for example, a method in which collagen gel is used for culturing in a medium containing EGF and insulin-like growth factor 2 (IGF2).

In some embodiments, differentiated CLiPs can form a bile duct-like structure. In a particular embodiment, the BEC induction method includes the steps of:

(i) culturing CLiPs on feeder cells at low density in the presence of inhibitor(s) and optionally serum; and

(ii) further culturing the cells obtained in step (i) in a medium containing Matrigel.

The feeder cells used in step (i) is not particularly limited and any cells that are generally used for the purpose of supporting maintenance and culture can be used. For example, they may be mouse fetal-derived fibroblasts (MEF) and STO cells (ATCC, CRL-1503), preferably MEF.

Low density refers to a density lower than the cell density generally used for the purpose of supporting maintenance and culture, which is, for example, a cell density in a range of 1×10³-5×10⁴ cells/cm², and preferably 5×10³-3×10⁴ cells/cm². A culture vessel for seeding the feeder cells may be one that is coated with a cell supporting substrate such as collagen or gelatin. The primary or passaged CLiPs can be treated with trypsin to be dissociated, resuspended in a medium containing inhibitor(s) and optionally serum, and seeded on the feeder cells at a cell density of 10⁴-10⁵ cells/cm². If necessary, the medium may be added with a serum.

On the following day, the medium can be replaced with a maintenance medium for pluripotent stem cells such as mTeSR™ (Stemcell Technologies), and subjected to culture in the presence of inhibitor(s) and optionally serum for 3-10 days, preferably 4-8 days. The medium can be freshly exchanged every 1-3 days. Subsequently, the medium can be exchanged with a medium containing Matrigel and further subjected to culture for 3-10 days, preferably 4-8 days. The medium can be freshly exchanged every 1-3 days. The concentration of the Matrigel added to the medium can suitably be selected in a range of 1-5%, preferably 1-3%. With a total of about 1-3 weeks of culture, a bile duct-like structure is formed where the cells are expressing CK19 and GRHL2 as BEC markers at high levels. Moreover, gene and protein expressions of aquaporins such as AQP1 and AQP9 and ion channels such as CFTR and AE2 are increased. In addition, strong expression of ZO-1 as a tight junction marker is observed in the lumen of the duct structure. Furthermore, since these cells have the ability of transporting water and the ability of transporting and accumulating drug metabolites in the lumen, LSC of the present invention can differentiate into functional BEC.

### B. Cell-Free Materials

In general, cell-based therapies can have limitations such as uncontrolled differentiation, side effects, tumor formation, and incompatibility of allogenic use. On the contrary, therapeutic and non-therapeutic uses of extracellular vesicles (EVs) from CLiPs have the possibility of overcoming such disadvantages. Thus, also provided are cell-free compositions derived from CLiPs.

Cell-free compositions including EVs and methods of use thereof are provided. The EVs can be part of a heterogeneous mixture of factors such as conditioned media, or a fraction isolated therefrom. In other embodiments, EVs, or one or more subtypes thereof, are isolated or otherwise collected from conditioned media of CLiPs. The EVs, or one or more subtypes thereof, can be suspended in a pharmaceutically acceptable composition, such as a carrier or matrix or depot, prior to administration to the subject.

### 1. Extracellular Vesicles

The disclosed compositions can be, or include, extracellular vesicles derived from CLiPs, or an isolated or fractionated subtype or subtypes thereof. Extracellular vesicles are lipid bilayer-delimited particles that are naturally released from a cell and, unlike a cell, cannot replicate. EVs range in diameter from near the size of the smallest physically possible unilamellar liposome (around 20-30 nanometers) to as large as 10 microns or more, although the vast majority of EVs are smaller than 200 nm.

Diverse EV subtypes have been proposed including ectosomes, microvesicles (MV), microparticles, exosomes, oncosomes, apoptotic bodies (AB), tunneling nanotubes (TNT), and more (Yáñez-Mó, et al., J Extracell Vesicles. 4:27066 (2015) doi:10.3402/jev.v4.27066. PMC 4433489). These EV subtypes have been defined by various, often overlapping, definitions, based mostly on biogenesis (cell pathway, cell or tissue identity, condition of origin) (Théry, et al., J Extracell Vesicles. 7 (1): 1535750 (2018). doi:10.1080/20013078.2018.1535750). However, EV subtypes may also be defined by size, constituent molecules, function, or method of separation. As discussed in Théry, et al., subtypes of EVs may be defined by:

a) physical characteristics of EVs, such as size (“small EVs” (sEVs) and “medium/large EVs” (m/1EVs), with ranges defined, for instance, respectively, <100 nm or <200 nm [small], or >200 nm [large and/or medium]) or density (low, middle, high, with each range defined);

b) biochemical composition (CD9+/CD63+/CD81+-EVs, Annexin A5-stained EVs, etc.); or

c) descriptions of conditions or cell of origin (podocyte EVs, hypoxic EVs, large oncosomes, apoptotic bodies).

Thus, in some embodiments, the composition is, or includes, one or more EV subtypes defined according (a), (b), or (c) as discussed above.

In some embodiments, the vesicles are, or include, exosomes. Exosomes possess surface proteins that promote endocytosis and they have the potential to deliver macromolecules. Also, if exosomes are obtained from the same individual as they are delivered to, the exosomes will be immunotolerant.

Exosomes are vesicles with the size of 30-150 nm, often 40-100 nm, and are observed in most cell types. Exosomes are often similar to MVs with an important difference: instead of originating directly from the plasma membrane, they are generated by inward budding into multivesicular bodies (MVBs). The formation of exosomes includes three different stages: (1) the formation of endocytic vesicles from plasma membrane, (2) the inward budding of the endosomal vesicle membrane resulting in MVBs that consist of intraluminal vesicles (ILVs), and (3) the fusion of these MVBs with the plasma membrane, which releases the vesicular contents, known as exosomes.

Exosomes have a lipid bilayer with an average thickness of ˜5 nm (see e.g., Li, Theranostics, 7(3):789-804 (2017) doi: 10.7150/thno.18133). The lipid components of exosomes include ceramide (sometimes used to differentiate exosomes from lysosomes), cholesterol, sphingolipids, and phosphoglycerides with long and saturated fatty-acyl chains. The outer surface of exosomes is typically rich in saccharide chains, such as mannose, polylactosamine, alpha-2,6 sialic acid, and N-linked glycans.

Many exosomes contain proteins such as platelet derived growth factor receptor, lactadherin, transmembrane proteins and lysosome associated membrane protein-2B, membrane transport and fusion proteins like annexins, flotillins, GTPases, heat shock proteins, tetraspanins, proteins involved in multivesicular body biogenesis, as well as lipid-related proteins and phospholipases. These characteristic proteins therefore serve as good biomarkers for the isolation and quantification of exosomes. Another key cargo that exosomes carry is nucleic acids including deoxynucleic acids (DNA), coding and non-coding ribonucleic acid (RNA) like messenger RNA (mRNA) and microRNA (miRNA).

In some embodiments, the vesicles include, or are, one or more alternative extracellular vesicles, such as ABs, MVs, TNTs, or others discussed herein or elsewhere.

ABs are heterogenous in size and originate from the plasma membrane. They can be released from all cell types and are about 1-5 μm in size

MVs with the size of 20 nm-1 μm are formed due to blebbing with incorporation of cytosolic proteins. In contrast to ABs, the shape of MVs is homogenous. They originate from the plasma membrane and are observed in most cell types.

TNTs are thin (e.g., 50-700 nm) and up to 100 μm long actin containing tubes formed from the plasma membrane.

In some embodiments, the EVs are between about 20 nm and about 500 nm. In some embodiments, the EVs are between about 20 nm and about 250 nm or 200 nm or 150 nm or 100 nm.

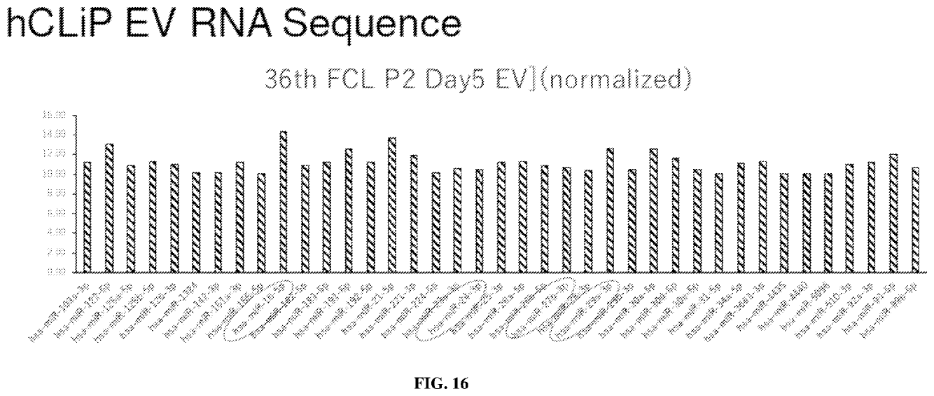

The Examples below show that EVs isolated from CLiPs include a number of miRNAs and cytokines. The Examples below show that EVs isolated from CLiPs include hsa-miR-103a-3p, hsa-miR-122-5p, hsa-miR-125a-5p, hsa-miR-125b-5p, hsa-miR-126-3p, hsa-miR-1324, hsa-miR-142-3p, hsa-miR-151a-3p, hsa-miR-155-5p, hsa-miR-16-5p, hsa-miR-182-5p, hsa-miR-183-5p, hsa-miR-191-5p, hsa-miR-192-5p, hsa-miR-21-5p, hsa-miR-221-3p, hsa-miR-224-5p, hsa-miR-23a-3p, hsa-miR-24-3p, hsa-miR-24-3p, hsa-miR-26a-3p, hsa-miR-28-3p, hsa-miR-29a-3p, hsa-miR-29b-3p, hsa-miR-30a-5p, hsa-miR-30d-5p, hsa-miR-30e-5p, hsa-miR-31-5p, hsa-miR-34a-5p, hsa-miR-3663-3p, hsa-miR-4435, hsa-miR-4440, hsa-miR-5096, hsa-miR-510-3p, hsa-miR-92a-3p, hsa-miR-93-5p, and hsa-miR-99b-5p (see, e.g., FIG. 16), and TNFα.

Thus, in some embodiments, the EVs include one or more of hsa-miR-103a-3p, hsa-miR-122-5p, hsa-miR-125a-5p, hsa-miR-125b-5p, hsa-miR-126-3p, hsa-miR-1324, hsa-miR-142-3p, hsa-miR-151a-3p, hsa-miR-155-5p, hsa-miR-16-5p, hsa-miR-182-5p, hsa-miR-183-5p, hsa-miR-191-5p, hsa-miR-192-5p, hsa-miR-21-5p, hsa-miR-221-3p, hsa-miR-224-5p, hsa-miR-23a-3p, hsa-miR-24-3p, hsa-miR-24-3p, hsa-miR-26a-3p, hsa-miR-28-3p, hsa-miR-29a-3p, hsa-miR-29b-3p, hsa-miR-30a-5p, hsa-miR-30d-5p, hsa-miR-30e-5p, hsa-miR-31-5p, hsa-miR-34a-5p, hsa-miR-3663-3p, hsa-miR-4435, hsa-miR-4440, hsa-miR-5096, hsa-miR-510-3p, hsa-miR-92a-3p, hsa-miR-93-5p, hsa-miR-99b-5p, and/or one or more cytokines such as TNFα, or any combination thereof.

### 2. Methods of Making Extracellular Vesicles

a. Sources of Cells for Making Extracellular Vesicles

As used herein, EVs, including AB, MV, exosomes, and TNT typically refers to lipid vesicles formed by cells or tissue. They can be isolated from tissue, cells, and/or fluid directly from a subject, including cultured and uncultured tissue, cells, or fluids, and fluid derived or conditioned by cultured cells (e.g., conditioned media). For example, exosomes are present in physiological fluids such as plasma, lymph liquid, malignant pleural effusion, amniotic liquid, breast milk, semen, saliva, and urine, and are secreted into the media of cultured cells.

The EVs of the disclosed compositions are typically formed from CLiPs. The CLiPs can be prepared and maintained as discussed above and below herein, or elsewhere, e.g., (Katsuda et al., Cell Stem Cell 20, 41-55, (2017), dx.doi.org/10.1016/j.stem.2016.10.007, Katsuda et al., eLife 8:e47313, 31 pages, (2019) doi.org/10.7554/eLife.47313, and U.S. Pat. No. 10,961,507, each of which is specifically incorporated by reference herein in its entirety).

Methods of isolating extracellular vesicles from tissue, cells, and fluid directly from a subject, including cultured and uncultured tissue, cells, or fluids, and fluid derived or conditioned by cultured cells (e.g., conditioned media) are known in the art.

See, for example, Li, Thernaostics, 7(3):789-804 (2017) doi: 10.7150/thno.18133, Ha, et al., Acta Pharmaceutica Sinica B, 6(4):287-296 (2016) doi: 10.1016/j.apsb.2016.02.001, Skotland, et al., Progress in Lipid Research, 66:30-41 (2017) doi: 10.1016/j.plipres.2017.03.001, Phinney and Pittenger, Stem Cells, 35:851-858 (2017) doi: 10.1002/stem.2575, each of which is specifically incorporated by reference, and describes isolating extracellular vesicles, particularly exosomes.

The EVs can be collected from primary cells or tissue or fluid. In some embodiments, the vesicles are isolated from cells, tissue, or fluid of the subject to be treated. An advantage of utilizing EVs that are isolated from natural sources includes avoidance of immunogenicity that can be associated with artificially produced lipid vesicles.

The EVs can also be collected from cell lines or tissue. Exemplary cells lines are commercially available and include those various sources including human bone-marrow, human umbilical cord, human embryonic tissue, and human adipose including those derived from lipoaspirate or dedifferentiated from mature adipocytes.

b. Methods of Collecting Extracellular Vesicles

Extracellular vesicles, including exosomes, can be isolated using differential centrifugation, flotation density gradient centrifugation, filtration, high performance liquid chromatography, and immunoaffinity-capture.

For example, one of the most common isolation techniques for isolating exosomes from cell culture is differential centrifugation, whereby large particles and cell debris in the culture medium are separated using centrifugal force between 200-100,000xg and the exosomes are separated from supernatant by the sedimenting exosomes at about 100,000xg. Purity can be improved, however, by centrifuging the samples using flotation density gradient centrifugation with sucrose or Optiprep. Tangential flow filtration combined with deuterium/sucrose-based density gradient ultracentrifugation was employed to isolate therapeutic exosomes for clinical trials.

In the Examples below, hCLiPs were suspended in SHM+FAC and seeded and cultured for 4 days. The last media exchange was serum-free. Culture supernatant was collected and centrifuged at 20000 g. The supernatant thereof was filtered and ultracentrifuged at 35000 rpm. After the ultracentrifugation, the supernatant was disposed of and the exosome was formed into a pellet (ultracentrifugation can be repeated depending on the amount of the culture supernatant). PBS was added to the pellet, the mixture thereof was ultracentrifuged again, the supernatant was disposed of, and the resulting product was washed. The pellet was dissolved with a very small amount of PBS (about 100 μL) left in the tube to prepare exosome solution.

Ultrafiltration and high performance liquid chromatography (HPLC) are additional methods of isolating EVs based on their size differences. EVs prepared by HPLC are highly purified.

Hydrostatic filtration dialysis has been used for isolating extracellular vesicles from urine.

Other common techniques for EV collection involve positive and/or negative selection using affinity-based methodology. Antibodies can be immobilized in different media conditions and combined with magnetic beads, chromatographic matrix, plates, and microfluidic devices for separation. For example, antibodies against exosome-associated antigens—such as cluster of differentiation (CD) molecules CD63, CD81, CD82, CD9, epithelial cell adhesion molecule (EpCAM), and Ras-related protein (Rab5)—can be used for affinity-based separation of exosomes. Non-exosomes vesicles that carry these or different antigens can also be isolated in a similar way.

Microfluidics-based devices have also been used to rapidly and efficiently isolate EVs such as exosomes, tapping on both the physical and biochemical properties of exosomes at microscales. In addition to size, density, and immunoaffinity, sorting mechanisms such as acoustic, electrophoretic and electromagnetic manipulations can be implemented.

Methods of characterizing EVs including exosomes are also known in the art. Exosomes can be characterized based on their size, protein content, and lipid content. Exosomes are sphere-shaped structures with sizes between 40-100 nm and are much smaller compared to other systems, such as a microvesicle, which has a size range from 100-500 nm. Several methods can be used to characterize EVs, including flow cytometry, nanoparticle tracking analysis, dynamic light scattering, western blot, mass spectrometry, and microscopy techniques. EVs can also be characterized and marked based on their protein compositions. For example, integrins and tetraspanins are two of the most abundant proteins found in exosomes. Other protein markers include TSG101, ALG-2 interacting protein X (ALIX), flotillin 1, and cell adhesion molecules. Similar to proteins, lipids are major components of EVs and can be utilized to characterize them.

### C. Pharmaceutical Compositions

Pharmaceutical compositions including CLiPs, EVs, and other molecules described herein for modulating liver function (e.g., one or more of the miRNAs or cytokines, or nucleic acids encoding the same, etc.) are also provided. Pharmaceutical compositions can be administered parenterally (e.g., intramuscular (IM), intraperitoneal (IP), intravenous (IV), subcutaneous (SubQ), or subdermal injection or infusion), transdermally (either passively or using iontophoresis or electroporation), or by any other suitable means, and can be formulated in dosage forms appropriate for each route of administration.

In some embodiments, the compositions are administered systemically, for example, by intravenous or intraperitoneal administration, in an amount effective for delivery of the compositions to targeted cells.

In some embodiments, the compositions are administered locally, for example, by injection directly into, or adjacent to, a site to be treated. For example, in some embodiments such as for the treatment of liver, the compositions are injected or otherwise administered directly to the liver or the area adjacent thereto, though other sites are also contemplated. For example, orthotopic liver transplantation is a therapeutic response for the treatment of several liver diseases, for example, hepatic cirrhosis, fulminant hepatitis, and several lethal hereditary enzyme deficiencies (Sharma, et al., Toxicologic Pathology, 40(1):83-92 (2012). doi:10.1177/0192623311425061). Although the procedure is now routine, it is not without its drawbacks, including post-transplantation complications as well as a shortage of donors. Accordingly, alternative procedures have been investigated to support liver function. Among these procedures is the transplantation of isolated hepatocytes to various systemic sites. Numerous sites have been examined, including fat pads, muscle, subcutaneous tissue, peritoneum, lungs, kidney, liver, and spleen. Thus, in some embodiments, the disclosed compositions include cells and/or cell-free materials are administered locally to one or more of the foregoing sites.

In some embodiments, local injection causes an increased localized concentration of the compositions which is greater than that which can be achieved by systemic administration.

In some embodiments, the compositions are delivered locally to the appropriate location by using a catheter or syringe. Other means of delivering such compositions locally include using infusion pumps (for example, from Alza Corporation, Palo Alto, Calif.) or incorporating the compositions into polymeric implants (see, for example, P. Johnson and J. G. Lloyd-Jones, eds., Drug Delivery Systems: Fundamentals and Techniques (Chichester, England: Ellis Horwood Ltd., 1988 ISBN-10:0895735806), which can effect a sustained release of the material to the immediate area of the implant.

The compositions can be provided to the cells either directly, such as by contacting it with the cells, or indirectly, such as through the action of any biological process. For example, the vesicles can be formulated in a physiologically acceptable carrier, and injected into a tissue or fluid surrounding the cells.

Exemplary dosages for in vivo methods are discussed in the experiments below. As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally, for local injection or infusion, dosage may be lower. Generally, the total amount of the active agent administered to an individual using the disclosed vesicles can be less than the amount of active agent that must be administered for the same desired or intended effect and/or may exhibit reduced toxicity.

In a preferred embodiment the compositions are administered in an aqueous solution, by parenteral injection such as intramuscular, intraperitoneal, intravenous, subcutaneous, subdermal, etc.

The formulation can be in the form of a suspension or emulsion. In general, pharmaceutical compositions are provided including effective amounts of one or more active agents optionally include pharmaceutically acceptable diluents, preservatives, solubilizers, emulsifiers, adjuvants and/or carriers. Such compositions can include diluents, sterile water, buffered saline of various buffer content (e.g., Tris-HCl, acetate, phosphate) at various pHs and ionic strengths; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

Transdermal formulations may also be prepared. These will typically be ointments, lotions, sprays, or patches, all of which can be prepared using standard technology. Transdermal formulations can include penetration enhancers. Chemical enhancers and physical methods including electroporation and microneedles can work in conjunction with this method.

### III. METHODS

The disclosed compositions can be used for treatment of liver diseases and disorders and injuries. The methods typically include administering a subject in need thereof one or more of the disclosed compositions in an effective amount to reduce or reverse one or more symptoms of liver disease or disorder, or liver damage.

Liver diseases and disorders include, but are not limited to, infections such as hepatitis A, B, and C; immune system problems such as autoimmune hepatitis, primary biliary cholangitis, and primary sclerosing cholangitis; cancers such as liver cancer, bile duct cancer, and liver cell adenoma; inherited liver disorders such as hemochromatosis, hyperoxaluria, Wilson's disease, and Alpha-1 antitrypsin deficiency; damage from alcohol abuse and/or drug overdose; nonalcoholic fatty liver disease. Dire complications of liver disease include acute liver failure and cirrhosis. In some embodiments, the liver disease or disorder is or includes hepatic fibrosis.

In some embodiments, the composition reduces existing hepatic fibrosis and/or the formation of new fibrosis. In some embodiments, the composition reduces the amount of existing hepatic collagen or the formation of new hepatic collagen (e.g., as measured by the amount of hydroxyproline). In some embodiments, the composition reduces the amount of existing fibrosis or the formation of new fibrosis as detected by staining with anti-Col1a antibody, a change in expression of one or more hepatic fibrosis-associated genes (e.g., increased expression of Mmp2 mRNA, reduced expression of Timp1, αSMA, and/or Col1a mRNA and/or protein, or any combination thereof).

In preferred embodiments, the composition induces a reduction in the expression of one or more markers of hepatic stellate cell activation, such as αSMA, preferrable in hepatic stellate cells

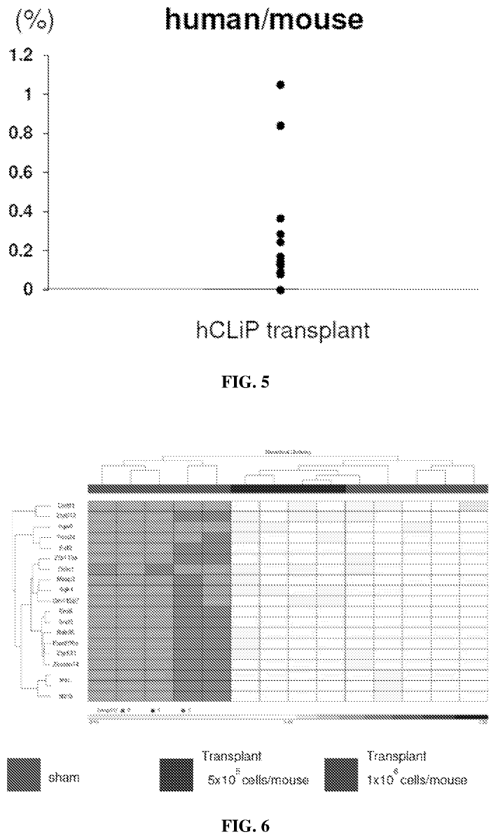

In some embodiments, the composition induces a change in expression of one or more genes associated with cell cycle, autophagy, cell membrane fusion, and/or zinc finger protein, such as Dmtf1, Zfp612, Itga6, Trim24, Eaf2, Zfp119a, Dido1, Masp2, Sgk1, Sm11567, Eml5, Srsf5, Rab35, Fam206a, Zfp131, Zkscan14, Insc, and Ntn3 (see, e.g., FIG. 6).

In some embodiments, the composition induces an increase in MMP1 and/or MMP13 mRNA and/or protein and/or a reduction in TNFα mRNA and/or protein in hepatic stellate cells.

The experimental results below show that TNFα can reduce expression of αSMA mRNA expression in hepatic stellate cells. Thus, in some embodiments, TNFα is included in the composition or otherwise co-administered with the disclosed compositions.

Compositions include, but not are not limited to, Clips, materials formed therefrom such as exosomes, and active elements thereof including but not limited to microRNA such as hsa-miR-103a-3p, hsa-miR-122-5p, hsa-miR-125a-5p, hsa-miR-125b-5p, hsa-miR-126-3p, hsa-miR-1324, hsa-miR-142-3p, hsa-miR-151a-3p, hsa-miR-155-5p, hsa-miR-16-5p, hsa-miR-182-5p, hsa-miR-183-5p, hsa-miR-191-5p, hsa-miR-192-5p, hsa-miR-21-5p, hsa-miR-221-3p, hsa-miR-224-5p, hsa-miR-23a-3p, hsa-miR-24-3p, hsa-miR-24-3p, hsa-miR-26a-3p, hsa-miR-28-3p, hsa-miR-29a-3p, hsa-miR-29b-3p, hsa-miR-30a-5p, hsa-miR-30d-5p, hsa-miR-30e-5p, hsa-miR-31-5p, hsa-miR-34a-5p, hsa-miR-3663-3p, hsa-miR-4435, hsa-miR-4440, hsa-miR-5096, hsa-miR-510-3p, hsa-miR-92a-3p, hsa-miR-93-5p, and/or hsa-miR-99b-5p, and/or one or more cytokines such as TNFα.

In the Examples below, some of the microRNA identified as present in exosomes produced by hCLiPs are classified by potential function/activity. Thus, in some embodiments, exosomes selected for a particular use may have one or more of the functions/activities desired for treatment of the target disease or disfunction as outlined:

Detected miRNAs were also classified by potential contribution of exosomal function/activity:
1) MicroRNA that acts for suppression of fibrosis
   miR-29b-3p: miR-29b-3p/HMGB1/TLR4/NF-κB signaling, αSMA↓
   miR-24,miR-27b: TGFbeta signaling↓
   miR-192-5p: Zeb1 and Zeb2 which are related to TGFbeta signaling;
Inhibition of EMT
2) MicroRNA that acts for hepatic regeneration:
   miR-24: cell growth & migration inhibition and promote differentiation;
Inhibition of TGFbeta signaling;
3) MicroRNAs having an anti-inflammatory action:
   miR-16: TNF↑: anti-apoptosis
4) MicroRNAs having a therapeutic effect on NASH:
   miR-182-5p; miR-183-5p
5) MicroRNAs having an effect of suppressing hepatoma:
   miR-23a; miR-27b, miR-31-5p; miR-182-5p; miR-183-5p

The compositions can be, for example, suspended in a suitable isotonic buffer (for example, PBS). Some embodiments, further include a pharmaceutically acceptable additive.

Although the suspension of e.g., cells or EVs may differ depending on the type of the liver disease, seriousness of the liver damage or the like, for example, 10⁸-10¹¹ cells can be transplanted by intraportal administration, intrasplenic administration or the like in a case of an adult.

Combination therapies are also contemplated. Thus, in some embodiments, CLiPs and/or EVs formed from CLiPs are co-administered to a subject in need thereof along with a second active agent. The second active agent can be in the same or different admixture with the CLiPs and/or EVs, and can be administered at the same or different times. In some embodiments, the additional active agent is a convention treatment for the disease or disorder (e.g., liver disease or disorder) from which the subject suffers.

### EXAMPLES

### Example 1: Hepatic Fibrosis is Improved by hCLiP Transplantation Materials and Methods

### Cells That Were Used

Primary human hepatocytes (Lot: FCL) were purchased from Veritas Corporation (Tokyo, Japan). Human Hepatic Stellate Cells (Science cell research laboratories) were purchased as hepatic stellate cells.

### Composition of the Medium

SHM was used as a basal medium for primary human hepatocytes. SHM was prepared by including 2.4 g/l NaHCO₃ and L-glutamine in DMEM/F12 (Life Technologies, MA) and adding 5 mM HEPES (Sigma, MO), 30 mg/l of L-proline (Sigma), 0.05% bovine serum albumin (Sigma), 10 ng/ml of epidermal growth factor (Sigma), insulin-transferrin-serine-X (Life Technologies), 10⁻⁷ M dexamethasone (Sigma), 10 mM nicotinamide (Sigma), 1 mM ascorbic acid-2 phosphate (Wako, Osaka, Japan), and antibiotic/antimycotic solution (Life Technologies) thereto. SHM+AC+10% FBS (SHM+FAC) prepared by adding 10% FBS (Life Technologies), 0.5 μM A-83-01 (Wako), and 3 μM CHIR99021 (Axon Medchem, Reston, VA) to this basal medium SHM was used for culturing hCLiPs. Stellate Cell Growth Supplement, 2% FBS, and P/S were each added to a Stellate Cell Medium (Science cell research laboratories) and used as a basal medium of hepatic stellate cells depending on the experiment.

Production of hCLiPs from Primary Human Hepatocytes

About a half of cryopreserved primary human hepatocytes were melt in a water bath at 37° C. and dissolved into 10 ml Leibovitz's L-15 Medium (Life Technologies) added with Glutamax (Life Technologies) and antibiotic/antimycotic solution. After 50 g of the mixture thereof was centrifuged for 5 minutes, a cell pellet was re-suspended in a William's E medium added with 10% FBS, GlutaMAX, antibiotic/antimycotic solution, and 10⁻⁷ M insulin (Sigma). Trypan blue (Life Technologies) was used to measure the number of living cells. Childhood primary human hepatocytes (Lot: FCL) were seeded on collagen I-coated plates (IWAKI, Shizuoka, Japan) at 2×10⁴ viable cells/cm². After 3-6 hours, the medium was exchanged with SHM+FAC. Subsequently, the medium was exchanged every 2-3 days and the cells were cultured for 14 days.

Subculture of hCLiPs

70-100% confluent hCLiPs were peeled off from the culture dish using TrypLE Express (Life Technologies, MA), and re-seeded on a 10 cm collagen-coated plate at 1×10⁵ cells/dish.

### Production of Model Mice With Hepatic Fibrosis

Carbon tetrachloride (0.5 ml/kg) was dissolved into olive oil at a ratio of 1:4 and intraperitoneally administered to 8-week-old NOD-SCID mice, which are immunodeficient mice, twice a week for 8 weeks, thereby causing hepatic fibrosis.

Transplantation of hCLiPs to Model Mice With Hepatic Fibrosis

The prepared hCLiPs were formed into a cell pellet by using TrypLE Express (Life Technologies, MA), which was then suspended in DMEM. The model mice with hepatic fibrosis that were being anesthetized with isoflurane were subjected to laparotomy, the spleen of the mice was exposed, and cell solution was injected at 5×10⁵ or 1×10⁶ cells/mouse, thereby intrasplenically transplanting the cells. After 2 weeks from the transplantation, the mice were dissected and the degree of hepatic fibrosis was evaluated.

### RNA Extraction

The total RNA was extracted by using a miRNeasy Mini Kit (QIAGEN, Venlo, The Netherlands).

### Reverse Transcription

For reverse transcription, a High-Capacity cDNA Reverse Transcription Kit (Life Technologies) was used.

### Real Time PCR

For cDNA, Real time-PCR was performed using Platinum SYBR Green qPCR SuperMix UDG (Lifetechnologies) or TaqMan™ Universal PCR Master Mix, no AmpErase™ UNG (Applied Biosystems). A change in gene expression was studied by using ACTB as an internal standard. The primers that were used are shown below in Table 1.

TABLE 1
Primer used in Real time PCR

| Gene | Forward | Reverse |
|---|---|---|
| Mmp2 | ACACTTTCTA | GTTTCAGGGT |
| | TGGCTGCCCC | CCAGGTCAGG |
| | (SEQ ID NO: 1) | (SEQ ID NO: 11) |
| Timp1 | GTAATGCGTC | GGGGGCCATC |
| | CAGGAAGCCT | ATGGTATCTG |
| | (SEQ ID NO: 2) | (SEQ ID NO: 12) |
| αSMA | GGCATCATCA | AGAGGCATAG |
| | CCAACTGGGA | AGGGACAGCA |
| | (SEQ ID NO: 3) | (SEQ ID NO: 13) |
| Col1a | TTCTCCTGGC | CTCAAGGTCA |
| | AAAGACGGAC | CGGTCACGAA |
| | (SEQ ID NO: 4) | (SEQ ID NO: 14) |
| β-actin | TCGTGCGTGA | GCCACAGGAT |
| | CATCAAAGAG | TCCATACCCA |
| | A | A |
| | (SEQ ID NO: 5) | (SEQ ID NO: 15) |
| MMP13 | TGGCTGCCTT | GAAAAGCATG |
| | CCTCTTCTTG | AGCCAGCAGG |
| | (SEQ ID NO: 6) | (SEQ ID NO: 16) |
| TNFα | CAGCTCCTAC | CTGGGCAGGT |
| | ATTGGGTCCC | CTACTTTGGG |
| | (SEQ ID NO: 7) | (SEQ ID NO: 17) |
| TIMP3 | ATGCCACCTC | ATTCTCCCCC |
| | CTGAGATCCT | TGCCAAATGG |
| | (SEQ ID NO: 8) | (SEQ ID NO: 18) |
| αSMA | CTGTTCCAGC | GGCAATGCCA |
| | CATCCTTCAT | GGGTACATAG |
| | (SEQ ID NO: 9) | (SEQ ID NO: 19) |
| β-ACTIN | AGCACTGTGT | ACTCTTCCAG |
| | TGGCGTACAG | CCTTCCTTCC |
| | (SEQ ID NO: 10) | (SEQ ID NO: 20) |

### MMP1:Gene Exp Mmp1 Hs00899658 M1 (Thermo Fisher)

### β-ACTIN: Gene Exp Actb Hs03023880 G1 (Thermo Fisher)

### Tissue Immunostaining

Antibodies that were used for tissue immunostaining are as follows. A paraffin block sample was made after fixation with formalin. After dewaxing it with ethanol and xylene, the antigen retrieval was performed using a solution prepared by diluting ImmunoSaver (Nissin EM, Tokyo, Japan) 200-fold at 98° C. for 45 minutes. Immersion in methanol comprising 0.3% H₂O₂ was performed at room temperature for 30 minutes to deactivate endogenous peroxidase. After infiltration with PBS comprising 0.1% Triton X-100, Blocking One solution was used to perform blocking at 4° C. for 30 minutes. Primary antibodies were then incubated at room temperature for 1 hour or at 4° C. overnight. The sample was stained using ImmPRESS IgG-peroxidase kits (Vector Labs, Burlingame, CA) and metal-enhanced DAB substrate kit (Life Technologies). Finally, the sample was immersed in hematoxylin solution and a cover glass was placed thereon to observe the sample.

Antibodies for immunohistochemistry

| Antibody | Host animal | Catalog # | Dilution | Manufacturer |
|---|---|---|---|---|
| Col1a | Goat | 1310-01 | 1/200 | Southern biotech |
| Mitochondria | Mouse | AB92824 | 1/1000 | Abcam |

### Digital PCR

The total DNA was collected from frozen liver tissue by using a DNeasy Blood & Tissue Kit (QIAGEN). The total DNA was used to detect human cells contained in the liver of the mice after transplanted with hCLiPs by QuantStudio™ 3D Digital PCR Master Mix v2 and Quant Studio 3D Digital PCR System (Thermo Fisher Scientific) by using a probe of Taqman Copy Number Reference Assay, Mousae Tfrc (VIC), and Taqman RNase P Detection Reagents Kit (FAM).

Hydroxyproline quantification

The liver tissue was used to quantify hydroxyproline by a Hydroxyproline Assay Kit (Bio Vsion).

### Results

### Production of Model Mice With Hepatic Fibrosis by Intraperitoneal Administration of Carbon Tetrachloride

Blood was collected by cutting the tail, the degree of fibrosis was monitored using AST and ALT, and conditions were studied for elapse of time after administration of carbon tetrachloride. For dosage, conditions were appropriately studied, with dosing of 100-400 mg/kg twice a week as a reference. While referring to prior study, 200 mg/kg of carbon tetrachloride was intraperitoneally administered to 6-week-old mice, which resulted in death of ⅓ mice in 10 days. All of the dead mice originally had a lower body weight or had a significantly reduced body weight after the administration. In view of these facts, it was considered that starting administration with 6-week-old mice was too early, so that the mice to start administration were changed to 8-week-old mice to study again. With administration starting with 8-week-old mice, only one mouse died, and stable production of mice with fibrosis succeeded. The degree of fibrosis was pathologically observed by sirius red staining or immunostaining using Col1a antibodies.

### Blood Biochemical Test of Model Mice With Hepatic Fibrosis

As of the transplantation, blood was collected by cutting the tail, serum was separated, and AST, ALT, total bilirubin, and platelet count were measured.

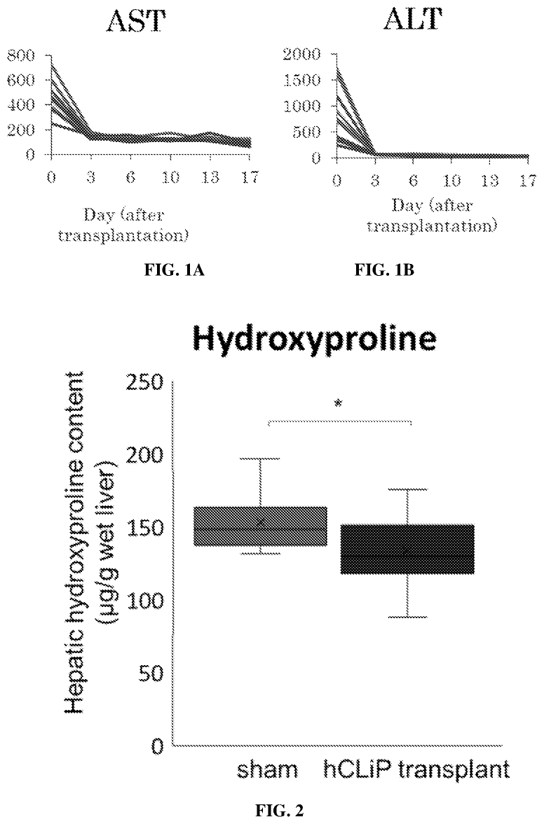

TABLE 2
Blood Biochemical Test Results (see also FIGS. 1A-1B).

| Blood biochemical test | Reference | | | | | |
|---|---|---|---|---|---|---|
| (as of transplantation) | value | #1 | #2 | #3 | #4 | #5 |
| Total bilirubin [mg/dL] | 0.2-1.2 | 0.1 | 0.1 | 0.1 | 0.1 | 0.1 |
| Platelet count [×10⁴/μL] | 14.0-37.9 | 46.1 | 136.9 | 125.3 | 79.7 | 92.8 |

Total bilirubin showed a normal value (Table 2). AST and ALT were measured over time twice a week, in which after they were significantly elevated at the initial dosing, they became stable and were higher than a reference value. These facts indicate that models with hepatic fibrosis at a mild level were produced. After the transplantation, the models were compared with the non-transplantation group with respect to AST/ALT, finding no significant difference (FIGS. 1A-1B).

Decrease in Collagen Amount in the Liver Tissue Due to hCLiP Transplantation

Hydroxyproline is one type of amino acid constituting collagen. Dissection was performed two weeks from hCLiP transplantation and the amount of hydroxyproline in the liver tissue was quantified. In the group transplanted with hCLiPs, the amount of hydroxyproline was significantly decreased (FIG. 2). This indicates the possibility that hCLiP transplantation suppresses collagen production or dissolves collagen.

Improvement in Pathological Hepatic Fibrosis Through hCLiP Transplantation

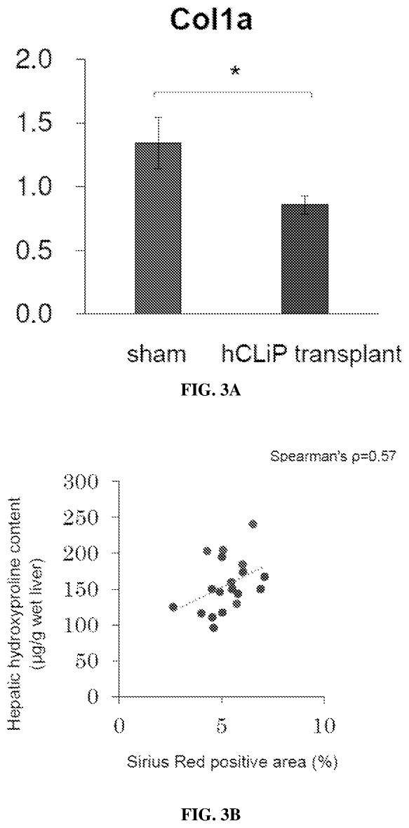

The degree of fibrosis was pathologically evaluated by sirius red staining or immunostaining using Collagen type 1a (Col1a) antibodies. In the group transplanted with hCLiPs, Col1a positive area was significantly reduced (FIG. 3A). With sirius red staining, a tendency to be improved by hCLiP transplantation was observed. In addition, it was revealed that sirius red staining and the amount of hydroxyproline show positive correlation (FIG. 3B).

Change in the Expression of the Hepatic Fibrosis-Associated Genes Due to hCLiP Transplantation

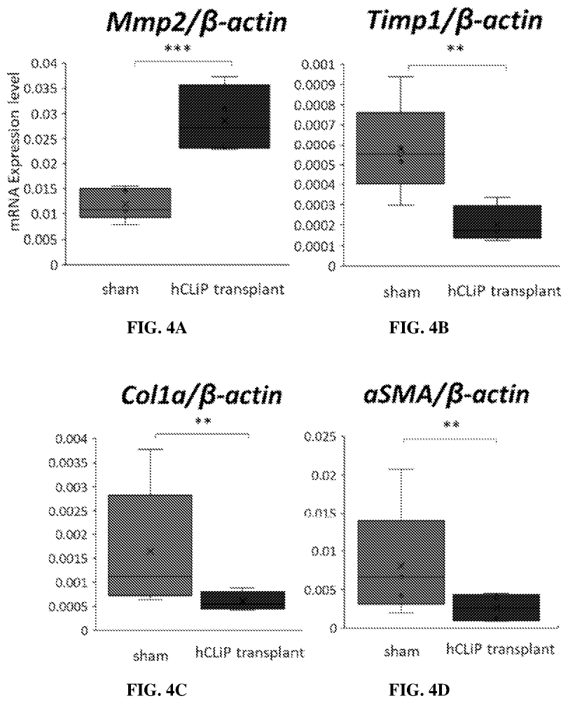

A change in expression of the genes (Mmp2, Timp1, αSMA, and Col1a) associated with hepatic fibrosis was observed with Real time-PCR. In the group transplanted with hCLiPs, the expression level of Mmp2 mRNA was significantly increased while the expression level of Timp1, αSMA, and Col1a mRNA was significantly decreased (FIGS. 4A-4D). As a result of hCLiP transplantation, increased expression of the gene dissolving collagen and decreased expression of the gene producing collagen were observed in the liver with fibrosis.

Presence of hCLiPs in the Liver Tissue

In order to study where the hCLiPs transplanted from the spleen are present, a liver tissue slice was used to perform immunostaining with human-mitochondria antibodies. However, detection failed. Thus, total DNA was collected from frozen liver tissue, and Mouse Tfrc and Human RNase P were used to measure the copy number of each of them. 0-1% of human cells were detected in the transplantation group (FIG. 5). Since there are 5×10⁵ transplanted cells and there are about 1×10⁸ cells in the murine liver, the number of transplanted hCLiPs/the number of cells in the murine liver is 0.5%. In week 2 after the transplantation, the percentage of human cells present in the murine liver was 1% at maximum, indicating that hCLiPs likely proliferated in the murine liver after the transplantation.

Change in the Gene Profile Due to hCLiP Transplantation

Microarray analysis was performed using the RNA of the non-plantation group and the plantation group. hCLiP transplantation resulted in a significant decrease in expression of 18 types of genes (Dmtf1, Zfp612, Itga6, Trim24, Eaf2, Zfp119a, Dido1, Masp2, Sgk1, Sm11567, Em15, Srsf5, Rab35, Fam206a, Zfp131, Zkscan14, Insc, and Ntn3) (FIG. 6). These are genes associated with cell cycle, autophagy, cell membrane fusion, or zinc finger protein, and their gene profile was greatly changed by hCLiP transplantation.

### Example 2: Co-Culture with Hepatic Stellate Cells Reveals the Therapeutic Mechanism of hCLiPs

### Materials and Methods

Co-Culture Using Hepatic Stellate Cells and hCLiPs

Hepatic stellate cells were suspended in a medium prepared by adding Stellate Cell Growth Supplement, 2% FBS, and P/S to a Stellate Cell Medium (Science cell research laboratories), and seeded at 1×10⁴ viable cells/cm². After overnight, the medium was exchanged with a medium prepared by adding TGFβ and P/S to a Stellate Cell Medium. After incubation for 24 hours, hCLiPs were suspended in a SHM medium, and co-culture was performed for 48 hours using a Transwell-COL insert (Corning) at 1×10⁵ viable cells/well.

### Addition of TNFα to Hepatic Stellate Cells

Hepatic stellate cells were suspended in a medium prepared by adding Stellate Cell Growth Supplement, 2% FBS, and P/S to a Stellate Cell Medium (Science cell research laboratories) and seeded at 1×10⁴ viable cells/cm². After overnight, the medium was exchanged with a medium prepared by adding TGFβ and P/S to a Stellate Cell Medium. After incubation for 24 hours, 5, 10, 20, 50 ng/ml of TNFα was added and exposed for 24 hours.

### Collection of Exosomes

hCLiPs were suspended in SHM+FAC and seeded at 2×10³ viable cells/cm². The medium was exchanged every 2 days, and the medium was exchanged with SHM+AC on day 4 of the culture. After culture for 24 hours, the culture supernatant was collected. The collected culture supernatant was centrifuged at 20000 g, at 4° C., for 10 minutes. The supernatant thereof was filtered with a Stericup Quick Release-GP Sterile Vacuum Filtration System (Millipore). The above processed culture supernatant was ultracentrifuged at 35000 rpm, at 4° C., for 1 hour and 10 minutes. Immediately after the ultracentrifugation, the supernatant was disposed of and the exosome was formed into a pellet (ultracentrifugation was repeated 2-5 times depending on the amount of the culture supernatant). PBS was added to the pellet, the mixture thereof was ultracentrifuged again, the supernatant was disposed of, and the resulting product was washed. The pellet was dissolved with a very small amount of PBS (about 100 μL) left in the tube to prepare exosome solution.

Analysis miRNA in hCLiP-Derived Exosomes

Collected exosomes were analyzed for the presences of miRNA. MiRNAs were purified from CLIP EVs by using Qiagen microRNAeasy kit. Purified microRNAs were put into Comprehensive miRNA expression analysis was performed using the 3D-Gene® miRNA Labeling kit and the 3D-Gene® Human miRNA Oligo Chip (both Toray Industries, Inc.), which was designed to detect 2,588 miRNA sequences registered in miRBase release 21 database (http://www.mirbase.org/). Microarray experiments were performed by Kamakura Techno-Science Inc. miRNAs with a signal intensity >2⁶ were considered detected miRNAs.

Addition of hCLiP-Derived Exosomes to Hepatic Stellate Cells

Hepatic stellate cells were suspended in a medium prepared by adding Stellate Cell Growth Supplement, 2% FBS, and P/S to a Stellate Cell Medium (Science cell research laboratories) and seeded at 1×10⁴ viable cells/cm². After overnight, the medium was exchanged with a medium prepared by adding TGFβ and P/S to a Stellate Cell Medium. After incubation for 24 hours, 10 μg/mL of hCLiP-derived exosome solution was added and the mixture thereof was cultured for 48 hours.

Production of immortalized hCLiPs

Three genes, CDK4, CCND1 (cyclin D1), and TERT, were introduced, and 4 types (A-D) of cells were created depending on a difference in the promoter.

| | |
|---|---|
| A | Expressed with CMV promoter |
| B | PGK promoter was used for only CCND1 while CMV promoter |
| | was used for the other two |
| C | TRE (tetracyclin responsive element) promoter + tetOff were |
| | expressed with CMV promoter |
| D | TRE (tetracyclin responsive element) promoter + tetOff were |
| | expressed with EpCAM promoter |

Induction of hepatic differentiation

hCLiPs were seeded on a collagen I-coated 24-well plate at a seeding density of 5×10⁴ cells/well (2.5×10⁴ cells/cm²). When it became 50-80% confluent, the medium was exchanged with SHM comprising 2% FBS, 0.5 mM A-83-01, and 3 mM CHIR99021. For a differentiation-induced group (Hep-i(+)), 5 ng/ml of human OSM (R&D) and 10⁻⁶ M dexamethasone were added. The cells were cultured for 6 days, in which the medium was exchanged every 2 days. On day 6, a mixture of Matrigel (Corning, Corning, NY) and the medium at a ratio of 1:7 was poured over the differentiation-induced group (Hep-i(+)) instead of the medium. On day 8, the gel was aspirated and washed with HANK's Balanced Salt Solution supplemented with Ca²⁺ and Mg²⁺ (Life Technologies).

Measurement of CYP activity

For measurement of CYP activity, SHM comprising 2% FBS was used as a basal medium. CYP3A4 was induced with 10 μM rifampicin or 1 mM phenobarbital. CYP1A2 was induced with 50 μM omeprazole. A medium comprising a CYP inducer was exchanged every day. After 3 days, a P450-Glo™ CYP3A4 Assay System (Promega) was used to measure CYP activity.

### Extraction of Protein

Cells were pipetted well with a M-PER™ Mammalian Protein Extraction Reagent and dissolved. The lysate was centrifuged at 15000 g, at 4° C., for 10 minutes, and the supernatant was used as protein solution. The protein concentration was measured using a Qubit®2.0 Fluorometer.

### Western Blotting

The protein solution was mixed with 4X SDS Sample Buffer (Merck), and the mixture thereof was incubated at 95° C. for 5 minutes to prepare a migration sample. Precision Plus Protein™ Dual Color Standards (BIORAD) were used as a molecular weight marker. 4-20% Mini-PROTEAN® TGX™ Precast Protein Gels (BIORAD) were placed in a migration tank, and the specimen and the molecular weight marker were applied. 100 ml 10× Tris/Glycine/SDS was diluted with 900 ml miliQ to be used as running buffer, and migration was performed at 100 V for 1 hour and 10 minutes. For transfer, 80 ml 10× Tris/Glycine was diluted with 720 ml miliQ and added with 200 ml methanol to be used as transfer buffer, and transfer to an immobilon-P membrane (Merck) was performed at 100 V for 1 hour. Blocking One solution was used to perform blocking at room temperature for 1 hour, primary antibodies were diluted with TBS-T added with 10% Blocking One solution, and they were left at 4° C. overnight. After the resulting product was washed with TBS-T three times, secondary antibodies were diluted with TBS-T and incubated at room temperature for 1 hour. The resulting product was washed with TBS-T three times again and stained with ImmunoStar LD (Wako, Japan), and detection was performed with a Molecular Imager ChemiDoc XRS System (BIORAD).

Antibodies for western blotting

| Antibody | Host animal | Catalog # | Dilution | Manufacturer |
|---|---|---|---|---|
| αSMA | Rabbit | 19245S | 1/1000 | CST |
| GAPDH | Mouse | MAB374 | 1/10000 | Millipore |

### Statistic Analysis

Statistic analysis was performed using SPSS. Student t test and Dunnett's test were performed. The notations of p<0.05: *, p<0,01: **, p<0.001: * will be hereinafter used.

### Results

Decrease in the Hepatic Stellate Cell Activation Level by Co-Culture of Hepatic Stellate Cells and hCLiPs

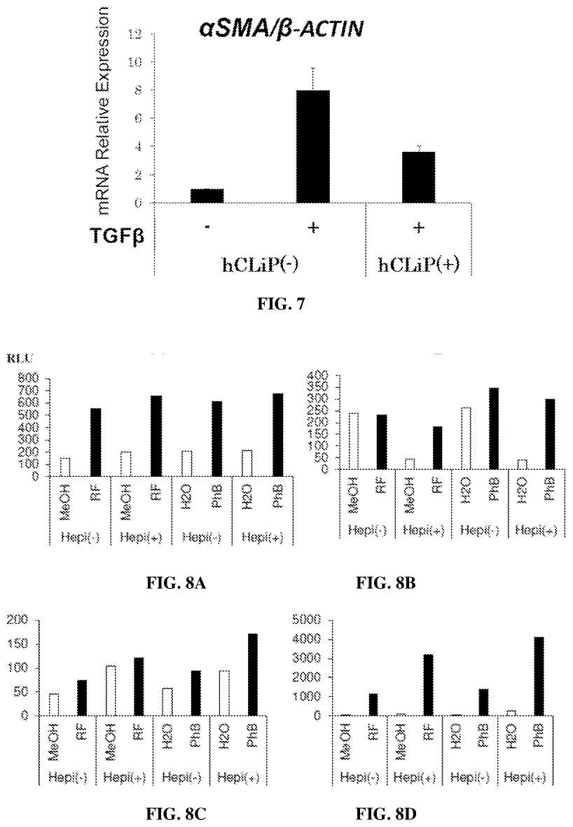

Hepatic stellate cells play a central role in the progression pathologic physiology of hepatic fibrosis. Activation of hepatic stellate cells causes hepatic stellate cells to produce extracellular matrix substance and play a central role in hepatic fibrosis. Thus, experiments were designed to investigate the impact of co-culture thereof with hCLiPs on hepatic stellate cell activation. Hepatic stellate cells were seeded, and after overnight, the medium was exchanged with a medium prepared by adding TGFβ and P/S to a Stellate Cell Medium. After incubation for 24 hours, hCLiPs were co-cultured for 48 hours using a Transwell-COL insert. As a result of co-culture of hepatic stellate cells and hCLiPs, expression of αSMA, which is a hepatic stellate cell activation marker, was significantly decreased at the mRNA and protein level in hepatic stellate cells (FIG. 7).

Decrease in the Hepatic Stellate Cell Activation Level by Co-Culture of Hepatic Stellate Cells and Immortalized hCLiPs

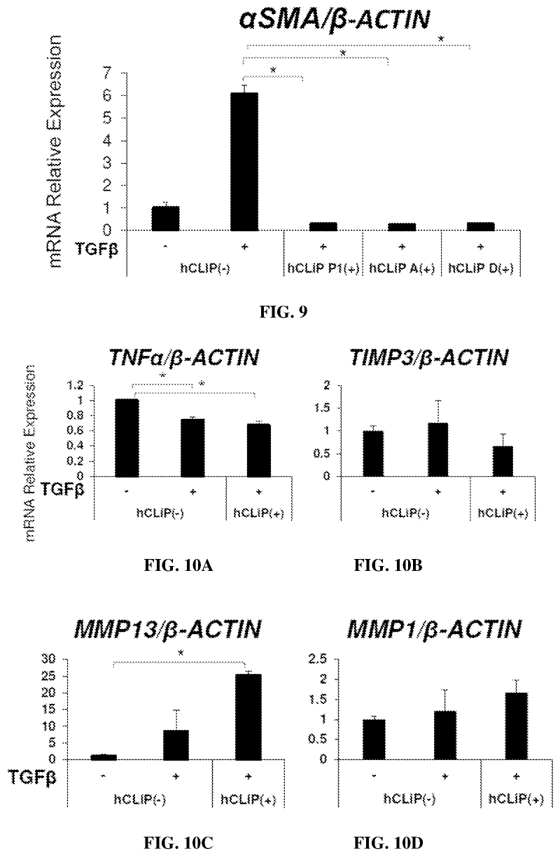

While hCLiPs have a significantly higher proliferation ability, the population of non-parenchymal cells is increased after repeated passages due to contamination of non-parenchymal cells. Thus, it is difficult to correctly evaluate the function and the therapeutic effect of hCLiPs after multiple passages. Thus, immortalized hCLiPs were produced to evaluate whether they have the same function as that of hCLiPs. First, four types (A-D) of immortalized hCLiPs were produced depending on a difference in the promoter or the like. In order to study whether the immortalized hCLiPs have the same function as that of hCLiPs, the immortalized hCLiPs were subjected to induction of differentiation and the CYP enzyme activity was measured. In A and D, the CYP enzyme activity was increased due to induction of differentiation (FIGS. 8A-8D), whereas in B and C, induction of differentiation caused no change and the enzyme activity was low. In view of these results, it is possible that other types of cells that were mixed such as bile duct epithelial cells were immortalized instead of hepatic progenitor cells in immortalization. Therefore, A and D were used as immortalized hCLiPs. Next, hepatic stellate cells and immortalized hCLiPs were co-cultured. As a result of co-culture of hepatic stellate cells and immortalized hCLiPs, expression of αSMA mRNA, which is a hepatic stellate cell activation marker, was significantly decreased in hepatic stellate cells (FIG. 9).

Change in the Gene Expression in Hepatic Stellate Cells Due to Co-Culture of Hepatic Stellate Cells and hCLiPs

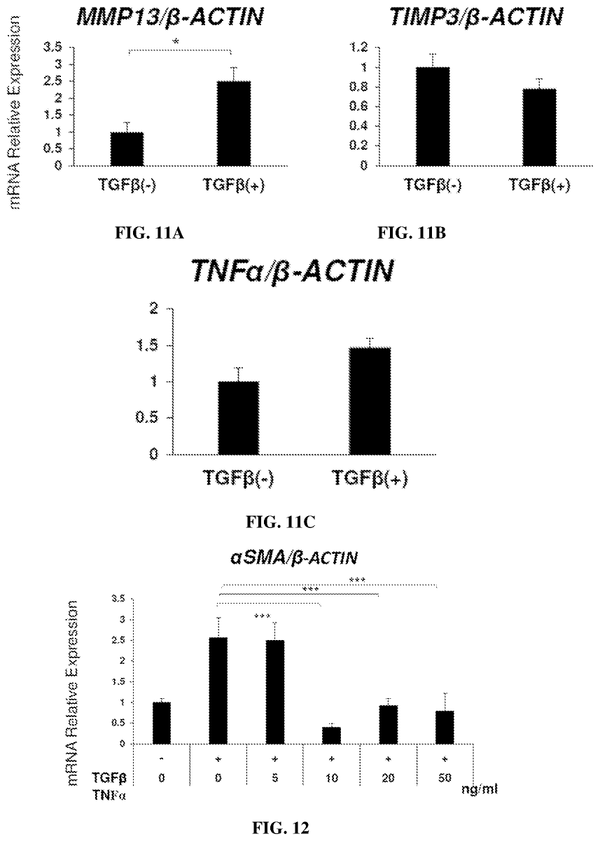

Hepatic stellate cells and hCLiPs were co-cultured to confirm the change in gene expression of signals involved in hepatic stellate cell activation and dissolution of collagen fibrosis. As a result of co-culture of hepatic stellate cells and hCLiPs, expression of MMP1 and MMP13 mRNA was increased in hepatic stellate cells. As a result of addition of TGFβ, expression of TNFα mRNA was decreased (FIGS. 10A-10D).

Change of hCLiPs Gene Expression Due to Co-Culture of Hepatic Stellate Cells and hCLiPs

Hepatic stellate cells and hCLiPs were co-cultured to confirm the change in gene expression of signals involved in hepatic stellate cell activation and dissolution of collagen fibrosis in the presence and absence of TGFβ. As a result of co-culture of hepatic stellate cells and hCLiPs in the presence of TGFβ, expression of MMP13 mRNA in hCLiPs was significantly increased. Expression of TNFα mRNA was increased, while expression of TIMP3 mRNA was decreased (FIGS. 11A-11C).

### Change in the Gene Expression Upon Addition of TNFαto Hepatic Stellate Cells

Since co-culture of hepatic stellate cells and hCLiPs in the presence of TGFβ resulted in an increase in the expression of TNFα mRNA in hCLiPs, it is believed that secretion of TNFα was increased from hCLiPs as a cytokine. Thus, TNFα, which is one type of cytokine, was added to activated hepatic stellate cells. Addition of TNFα resulted in a significant decrease in the αSMA mRNA expression in the 10, 20, and 50 ng/ml groups (FIG. 12).

Decrease in the Hepatic Stellate Cell Activation Level of When hCLiP-Derived Exosomes are Added to Hepatic Stellate Cells

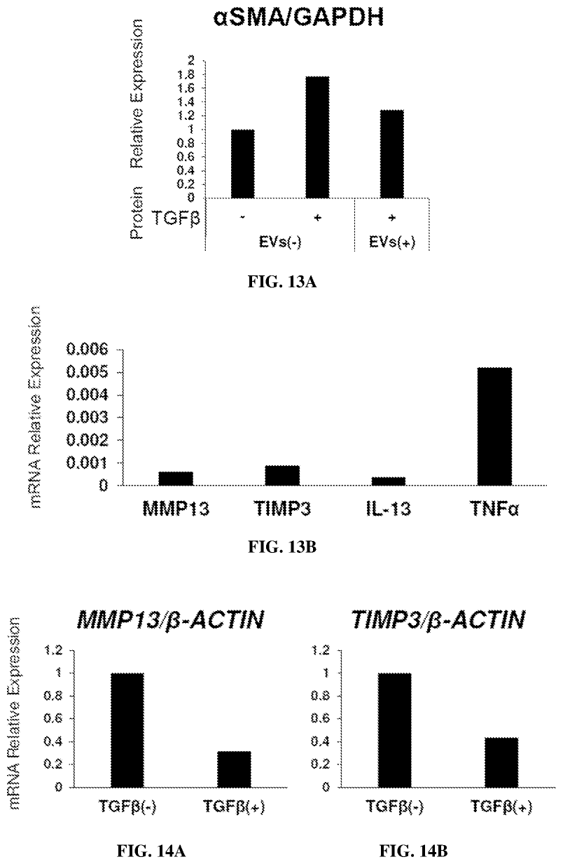

Co-culture of hepatic stellate cells and hCLiPs indicate a decrease in the expression of αSMA due to a hCLiP-derived secretion. There are various cell-derived secretions such as cytokines or exosomes. Exosomes are stable and easy use for cell-free therapy. Thus, hCLiP-derived exosomes were collected and added the exosome solution to hepatic stellate cells, and changes in expression were observed. Hepatic stellate cells were seeded, and after overnight, the medium was exchanged with a medium prepared by adding TGFβ and P/S to a Stellate Cell Medium. After incubation for 24 hours, 10 μg/mL of hCLiP-derived exosome solution was added, and the mixture thereof was cultured for 48 hours. Addition of a hCLiP-derived exosomes to hepatic stellate cells resulted in a decrease in the expression of αSMA, which is a hepatic stellate cell activation marker, at the protein level in hepatic stellate cells. Further, the gene expression of mRNA in the added exosome was confirmed, finding that many TNFα mRNAs were contained (FIGS. 13A-13B).

mRNA in hCLiP-Derived Exosomes in the Presence of TGFβ

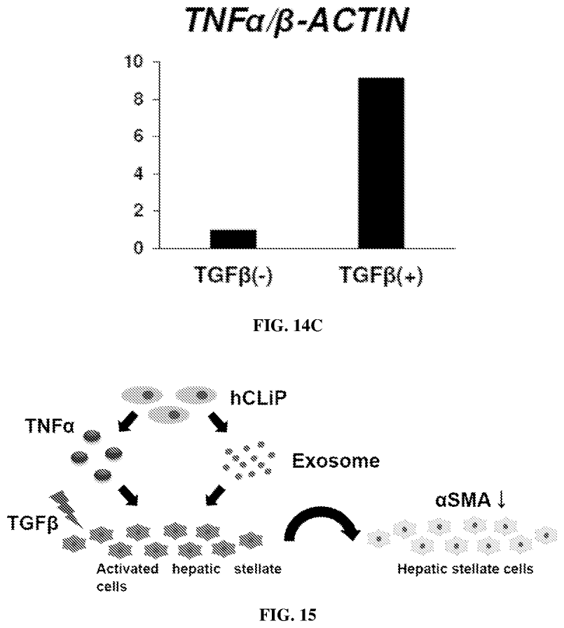

The exosomes were collected in the presence and absence of TGFβ, and a change in gene expression in the exosomes was observed. In the presence of TGFβ, the expression level of MMP13 and TIMP3 mRNA in the exosome was decreased. Further, the expression level of TNFα in the exosomes was increased (FIGS. 14A-14C).

miRNA in hCLiP-Derived Exosomes

Collected exosomes were also analyzed for the presences of miRNA. Results are shown in the FIG. 16. Circled miRNA are those believed to contribute to inhibition of fibrosis.

Detected miRNAs were also classified by potential contribution of exosomal function/activity:
1) MicroRNA that acts for suppression of fibrosis
   miR-29b-3p: miR-29b-3p/HMGB1/TLR4/NF-κB signaling, aSMA↓
   miR-24, miR-27b: TGFbeta signaling↓
   miR-192-5p: Zeb1 and Zeb2 which are related to TGFbeta signaling;
Inhibition of EMT
2) MicroRNA that acts for hepatic regeneration:
   miR-24: cell growth & migration inhibition and promote differentiation;
Inhibition of TGFbeta signaling;
3) MicroRNAs having an anti-inflammatory action:
   miR-16: TNF↑; anti-apoptosis
4) MicroRNAs having a therapeutic effect on NASH:
   miR-182-5p; miR-183-5p
5) MicroRNAs having an effect of suppressing hepatoma:
   miR-23a; miR-27b, miR-31-5p; miR-182-5p; miR-183-5p

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of making extracellular vesicles (EVs) comprising culturing chemically induced liver progenitor cells (CLiPs) and harvesting EVs secreted by the CLiPs.

2. The method of claim 1, wherein the CLiPs are formed by a method comprising culturing hepatocytes with an inhibitor of TGFβ signaling.

3. The method of claim 2, wherein the inhibitor of TGFβ signaling is A83-01.

4. The method of claim 3, wherein the A83-01 is in a concentration of about 1 μM to about 10 μM, or about 0.1 μM to about 10 μM, or about 0.5 μM.

5. The method of claim 1, wherein the CLiPs are formed by a method comprising culturing hepatocytes with a GSK3 inhibitor.

6. The method of claim 5, wherein the GSK3 inhibitor is CHIR99021.

7. The method of claim 6, wherein the CHIR99021 is in a concentration of about 0.1 μM to about 20 μM, about 1 μM to about 10 μM, or about 3 μM.

8. The method of claim 1, wherein the CLiPs are formed by a method comprising culturing hepatocytes with serum.

9. The method of claim 8, wherein the serum is Fetal Bovine Serum (FBS).

10. The method of claim 8 wherein the serum is about 5-20% of the culture media, or about 10% of the culture media.

11. The method of claim 1, wherein the CLiPs are formed by a method comprising culturing hepatocytes with a ROCK inhibitor.

12. The method of claim 11, wherein the ROCK inhibitor is Y-27632.

13. The method of claim 12, wherein the Y-27632 is in a concentration of about 1 μM to about 100 μM, or about 5 μM to about 25 μM, or about 10 μM.

14. The method of claim 1, wherein the cells began as hepatocytes isolated/purified from a liver of a mammal.

15. The method of claim 1, wherein the cells are cultured with the inhibitor(s) and/or serum for at least 5, 6, 7, 8, 9, 10, 11, 12, 13, 14, 15, 16, 17, 18, 19, or 20 days; or about 5 days to about 25 days, or any subrange or integer number of days therebetween, optionally for about 7 days to about 22 days, about 5 days to about 25 days, or about 10 days to about 20 days, or about 12 days to about 17 days; or about 13, 14, or 15 days.

16. The method of claim 1, wherein the EVs comprise or consists of exosomes.

17. The method of claim 1, wherein the cells are human cells and the culturing comprises TGBβ inhibitor, GSK3 inhibitor, and serum, and optionally excludes ROCK inhibitor.

18. The method of claim 1, wherein the cells are mouse or rat cells and the culturing comprises TGBβ inhibitor, GSK3 inhibitor, and ROCK inhibitor, and optionally excludes serum.

19. Extracellular vesicles (EVs) made according to the method of claim 1.

20. A pharmaceutical composition comprising an effective amount of the EVs of claim 19.

21. A therapeutic or non-therapeutic method of treating a subject, comprising administering the subject the pharmaceutical composition of claim 20.

22. A method of treating a subject for liver fibrosis comprising administering the subject a pharmaceutical composition comprising an effective amount of CLiPs or the pharmaceutical composition of claim 20.

23. The method of claim 21, wherein the CLiPs are formed from human cells.

24. The method of claim 22, wherein the pharmaceutical composition comprises CLiPs.

25. The method of claim 24, wherein the CLiPs secrete EVs comprising one or more of hsa-miR-103a-3p, hsa-miR-122-5p, hsa-miR-125a-5p, hsa-miR-125b-5p, hsa-miR-126-3p, hsa-miR-1324, hsa-miR-142-3p, hsa-miR-151a-3p, hsa-miR-155-5p, hsa-miR-16-5p, hsa-miR-182-5p, hsa-miR-183-5p, hsa-miR-191-5p, hsa-miR-192-5p, hsa-miR-21-5p, hsa-miR-221-3p, hsa-miR-224-5p, hsa-miR-23a-3p, hsa-miR-24-3p, hsa-miR-24-3p, hsa-miR-26a-3p, hsa-miR-28-3p, hsa-miR-29a-3p, hsa-miR-29b-3p, hsa-miR-30a-5p, hsa-miR-30d-5p, hsa-miR-30e-5p, hsa-miR-31-5p, hsa-miR-34a-5p, hsa-miR-3663-3p, hsa-miR-4435, hsa-miR-4440, hsa-miR-5096, hsa-miR-510-3p, hsa-miR-92a-3p, hsa-miR-93-5p, and hsa-miR-99b-5p, and/or one or more cytokines optionally wherein the cytokine(s) is or comprises TNFα, or any combination thereof.

26. The method of claim 21, wherein the pharmaceutical composition is cell-free.

27. The method of claim 21, wherein the pharmaceutical composition comprises EVs comprising one or more of hsa-miR-103a-3p, hsa-miR-122-5p, hsa-miR-125a-5p, hsa-miR-125b-5p, hsa-miR-126-3p, hsa-miR-1324, hsa-miR-142-3p, hsa-miR-151a-3p, hsa-miR-155-5p, hsa-miR-16-5p, hsa-miR-182-5p, hsa-miR-183-5p, hsa-miR-191-5p, hsa-miR-192-5p, hsa-miR-21-5p, hsa-miR-221-3p, hsa-miR-224-5p, hsa-miR-23a-3p, hsa-miR-24-3p, hsa-miR-24-3p, hsa-miR-26a-3p, hsa-miR-28-3p, hsa-miR-29a-3p, hsa-miR-29b-3p, hsa-miR-30a-5p, hsa-miR-30d-5p, hsa-miR-30e-5p, hsa-miR-31-5p, hsa-miR-34a-5p, hsa-miR-3663-3p, hsa-miR-4435, hsa-miR-4440, hsa-miR-5096, hsa-miR-510-3p, hsa-miR-92a-3p, hsa-miR-93-5p, and hsa-miR-99b-5p, and/or one or more cytokines optionally wherein the cytokine(s) is or comprises TNFα, or any combination thereof.

28. The method of claim 20 further comprising administering the subject TNFα.

29. The method of claim 20, wherein the subject has a liver disease or disorder.

30. The method of claim 29, wherein the liver disease or disorder is selected from an infection, optionally hepatitis A, B, or C; an immune system problem, optionally autoimmune hepatitis, primary biliary cholangitis, or primary sclerosing cholangitis; a cancer optionally liver cancer, bile duct cancer, or liver cell adenoma; an inherited liver disorder, optionally hemochromatosis, hyperoxaluria, Wilson's disease, or Alpha-1 antitrypsin deficiency;
damage from alcohol abuse and/or drug overdose; or nonalcoholic fatty liver disease.

31. The method of claim 22, wherein the CLiPs or EVs can reduce the amount of existing hepatic collagen or the formation of new hepatic collagen; reduce the amount of existing fibrosis or the formation of new fibrosis; induce a change in expression of one or more hepatic fibrosis-associated genes, optionally increased expression of Mmp2 mRNA, reduced expression of Timp1, αSMA, and/or Col1a mRNA and/or protein, or any combination thereof; induce a reduction in the expression of one or more markers of hepatic stellate cell activation, such as αSMA, preferrable in hepatic stellate cells; induce a change in expression of one or more genes associated with cell cycle, autophagy, cell membrane fusion, and/or zinc finger protein, optionally wherein the gene(s) is Dmtf1, Zfp612, Itga6, Trim24, Eaf2, Zfp119a, Dido1, Masp2, Sgk1, Sm11567, Em15, Srsf5, Rab35, Fam206a, Zfp131, Zkscan14, Insc, Ntn3, or a combination thereof; induce an increase in MMP1 and/or MMP13 mRNA and/or protein in hepatic stellate cells; and/or induce a reduction in TNFα mRNA and/or protein in hepatic stellate cells.
